(19) United States
(12) Patent Application Publication
(10) Pub. No.: US 2026/0356376 A1
(43) Pub. Date: Nov. 12, 2026

(54) GAMIFICATION OF INTOXICATION MONITORING SYSTEM

(71) Applicant: Consumer Safety Technology, LLC, Urbandale, IA (US)

(72) Inventors: Brian Clark, Ankeny, IA (US); David Livingston Smith, Des Moines, IA (US); James Braunschweig, Mitchellville, IA (US); Thomas Ryan Evans, Sherwood, OR (US)

(21) Appl. No.: 19/671,670

(22) Filed: May 8, 2026

Related U.S. Application Data
(60) Provisional application No. 63/802,935, filed on May 9, 2025.

(51) Int. Cl. B60W 50/00; A61B 5/00; B60W 50/14; G06Q 30/0207

(52) U.S. Cl. CPC B60W 50/0098; A61B 5/4845; A61B 5/742; B60W 50/14; G06Q 30/0207; A61B 2503/22; B60W 2050/146; B60W 2510/242; B60W 2520/10; B60W 2540/24

(57) ABSTRACT

An intoxication monitoring system can include an intoxication monitoring device configured to receive and transmit data regarding a user's actions and a processor performing the steps of receiving the data regarding the user's actions, wherein the data identifies a user account associated with the intoxication monitoring device and analyzing the user action by applying a reward matrix criteria. The steps can further include changing a point total associated with the user's account when the user's action meets the reward matrix criteria, displaying, on a user interface, one or more redemption notifications selectable by the user when the point total surpasses a redemption threshold, receiving the user's redemption selection, and processing the redemption selection.

This application claims the benefit of U.S. Provisional Application No. 63/802,935, filed May 9, 2025, the content of which is incorporated herein by reference in its entirety.

### FIELD

Embodiments herein relate to intoxication monitoring systems.

### BACKGROUND

Intoxication monitoring devices, such as breath alcohol ignition interlock systems, are widely used for a variety of purposes such as preventing impaired driving, ensuring compliance with court-ordered sobriety programs, and providing voluntary monitoring. However, the effectiveness of these devices relies heavily on proper use and maintenance by the users. Inconsistent or improper usage can lead to inaccurate readings, device malfunctions, and reduced overall effectiveness of the monitoring program.

Traditional approaches to encouraging proper device usage have often relied on punitive measures or the threat of legal consequences. While these methods can be effective to some degree, they may not always foster a positive attitude towards the monitoring program or encourage long-term behavioral changes. There is a need for innovative approaches that can motivate users to consistently and correctly use their intoxication monitoring devices, thereby improving the reliability and effectiveness of these systems.

### SUMMARY

In a first aspect, an intoxication monitoring system can be included, the method including an intoxication monitoring device configured to receive and transmit data regarding a user's actions, non-transitory computer-readable instructions executable by a processor, wherein execution of the instructions performs a method. The method can include receiving the data regarding the user's actions, wherein the data identifies a user account associated with the intoxication monitoring device, analyzing the user action by applying a reward matrix criteria, changing a point total associated with the user's account when the user's action meets the reward matrix criteria, displaying, on a user interface, one or more redemption notifications selectable by the user when the point total surpasses a redemption threshold, receiving the user's redemption selection, and processing the redemption selection.

In a second aspect, in addition to one or more of the preceding or following aspects, or in the alternative to some aspects, the intoxication monitoring device can be an interlock device.

In a third aspect, in addition to one or more of the preceding or following aspects, or in the alternative to some aspects, the user interface can be the user interface of a mobile device.

In a fourth aspect, in addition to one or more of the preceding or following aspects, or in the alternative to some aspects, applying the reward matrix criteria includes comparing elements of the reward matrix criteria with the user's actions to determine if the elements of the reward matrix criteria have been fulfilled.

In a fifth aspect, in addition to one or more of the preceding or following aspects, or in the alternative to some aspects, the user's action meets the reward matrix criteria if the user's action includes maintaining a suitable vehicle battery level for a specific amount of time or checking the intoxication monitoring device prior to exiting a vehicle to determine if a retest can be required.

In a sixth aspect, in addition to one or more of the preceding or following aspects, or in the alternative to some aspects, the user's action meets the reward matrix criteria if the user's action includes maintaining a suitable vehicle battery level for a specified time period.

In a seventh aspect, in addition to one or more of the preceding or following aspects, or in the alternative to some aspects, the vehicle battery level can be considered suitable if the vehicle runs for at least 15 minutes every other day or if the vehicle battery can be attached to a charger.

In an eighth aspect, in addition to one or more of the preceding or following aspects, or in the alternative to some aspects, the data transmitted from the intoxication monitoring device includes the user's vehicular speed, location, vehicle battery voltage, and test results.

In a ninth aspect, in addition to one or more of the preceding or following aspects, or in the alternative to some aspects, the user's action meets the reward matrix criteria, if the user's action includes demonstrating proficiency in the intoxication monitoring device's required breath pattern, calibrating of the intoxication monitoring device within a specified time period, providing a passing sample to the intoxication monitoring device, utilizing the intoxication monitoring device beyond any legal requirements to do so, using the user interface self-reporting information, or demonstrating continuous use of the user interface.

In a tenth aspect, in addition to one or more of the preceding or following aspects, or in the alternative to some aspects, the intoxication monitoring device can be a handheld monitoring device.

In an eleventh aspect, in addition to one or more of the preceding or following aspects, or in the alternative to some aspects, the user interface provides a notification to the user if the user's action does not meet the reward matrix criteria, wherein the notification includes a recommendation to correct the user's action.

In a twelfth aspect, in addition to one or more of the preceding or following aspects, or in the alternative to some aspects, the data transmitted from the intoxication monitoring device includes the user's test results and location.

In a thirteenth aspect, in addition to one or more of the preceding or following aspects, or in the alternative to some aspects, changing a point total associated with the user's account includes increasing the user's point total by one or more points.

In a fourteenth aspect, in addition to one or more of the preceding or following aspects, or in the alternative to some aspects, the method further includes changing the point total associated with the user's account by decreasing the user's point total by one or more points in response to a redemption selection.

In a fifteenth aspect, in addition to one or more of the preceding or following aspects, or in the alternative to some aspects, the user interface can be configured to display the user's total points.

In a sixteenth aspect, in addition to one or more of the preceding or following aspects, or in the alternative to some aspects, the user's total points can be indicative of a user's status level.

In a seventeenth aspect, in addition to one or more of the preceding or following aspects, or in the alternative to some aspects, the redemption notification can be indicative of the user's status level.

In an eighteenth aspect, in addition to one or more of the preceding or following aspects, or in the alternative to some aspects, the redemption notification includes at least one of free roadside service, free intoxication monitoring device insurance, free accessories for the intoxication monitoring device, a bill credit, a gift card, free uninstallation of the intoxication monitoring device, or upgrades to the user interface.

In a nineteenth aspect, a method of interacting with a user of an intoxication system to support reliable function includes receiving, from an intoxication monitoring device, a data regarding the user's action, wherein the data identifies a user account associated with the intoxication monitoring device, analyzing, using a mobile device, the user's action by applying a reward matrix criteria, changing a point total associated with the user's account if the user's actions meet the reward matrix criteria, displaying, on the user interface of the mobile device, one or more redemption notifications selectable by the user once the point total can be above a points threshold, receiving the user's redemption selection, and processing the redemption selection.

In a twentieth aspect, an intoxication monitoring system can include an intoxication monitoring device configured to receive and transmit data regarding a user's action and a mobile device in electronic communication with the intoxication monitoring device. The mobile device can include a user interface, a processor, a memory coupled to the processor, and non-transitory computer-readable instructions executable by the processor, wherein execution of the instructions causes the mobile device to perform a method. The method can include receiving the data regarding the user's action, wherein the data identifies a user account associated with the intoxication monitoring device, analyzing the user's action by applying a reward matrix criteria, changing a point total associated with the user's account if the user's action meet the reward matrix criteria, displaying, on the user interface, one or more notifications indicating an increase in a status level of the user once enough points can be received.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an intoxication monitoring system in accordance with various embodiments herein.

FIG. 2 is a schematic view of an intoxication monitoring system in accordance with various embodiments herein.

FIG. 3 is a schematic view of a user interface in accordance with various embodiments herein.

FIG. 4 is a schematic view of a user interface in accordance with various embodiments herein.

FIG. 5 is a schematic view of various components of an intoxication monitoring system in accordance with various embodiments herein.

FIG. 6 is a schematic view of a device log in accordance with various embodiments herein.

FIG. 7 is a schematic view of a device log in accordance with various embodiments herein.

FIG. 8 is a schematic view of a device log in accordance with various embodiments herein.

FIG. 9 is a processing scheme for analyzing device logs in accordance with various embodiments herein.

FIG. 10 is a processing scheme for analyzing device logs in accordance with various embodiments herein.

FIG. 11 is a processing scheme for analyzing device logs in accordance with various embodiments herein.

FIG. 12 is a schematic view of reward matrix criteria in accordance with various embodiments herein.

FIG. 13 is a schematic view of reward matrix criteria in accordance with various embodiments herein.

FIG. 14 is a graph depicting a user's accumulated points over time in accordance with various embodiments herein.

FIG. 15 is a graph depicting a user's accumulated points over time in accordance with various embodiments herein.

FIG. 16 is a computer system in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

The present disclosure provides an innovative approach embodied in a system, method, and device designed to enhance the usage and maintenance of intoxication monitoring systems through the implementation of gamification techniques. The disclosed subject matter aims to address the significant challenge of ensuring user compliance and engagement with intoxication monitoring systems, which traditionally rely on punitive measures to deter non-compliance. By integrating gamification principles, the intoxication monitoring system provides a positive reinforcement mechanism to motivate users to adhere to proper usage protocols and maintain device functionality, thereby improving reliability and user cooperation.

In various embodiments, an intoxication monitoring system can include an intoxication monitoring device configured to receive and transmit data regarding a user's actions. The intoxication monitoring system can further include non-transitory computer-readable instructions executable by a processor. In various embodiments, the execution of the instructions performs a method including the steps of receiving the data regarding the user's actions, wherein the data identifies a user account associated with the intoxication monitoring device, analyzing the user action by applying a reward matrix criteria, changing a point total associated with the user's account when the user's action meets the reward matrix criteria, displaying, on a user interface, one or more redemption notifications selectable by the user when the point total surpasses a redemption threshold, receiving the user's redemption selection, and processing the redemption selection.

It should be understood that the described embodiments are presented for purposes of illustration and description and are not intended to limit the scope of the disclosure. Various modifications, substitutions, and alterations may be made to the described embodiments without departing from the scope of the disclosure.

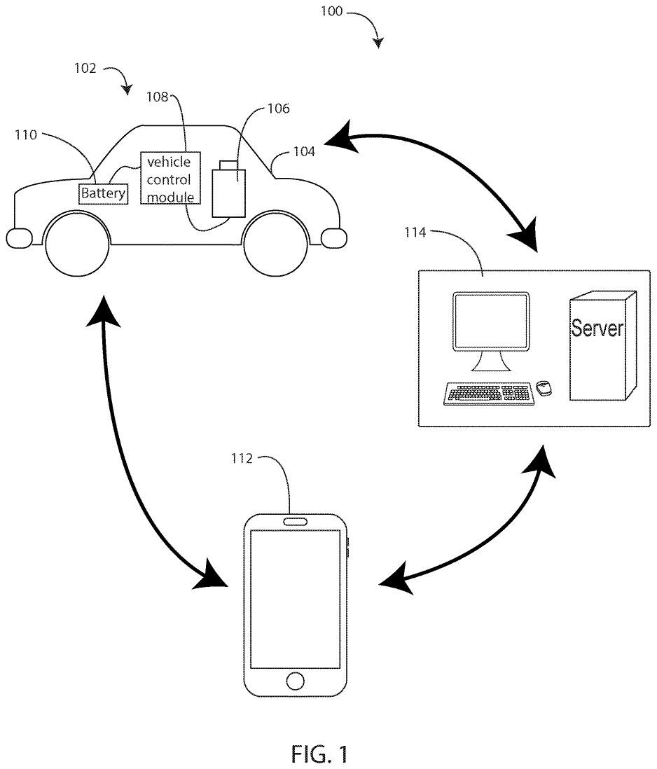

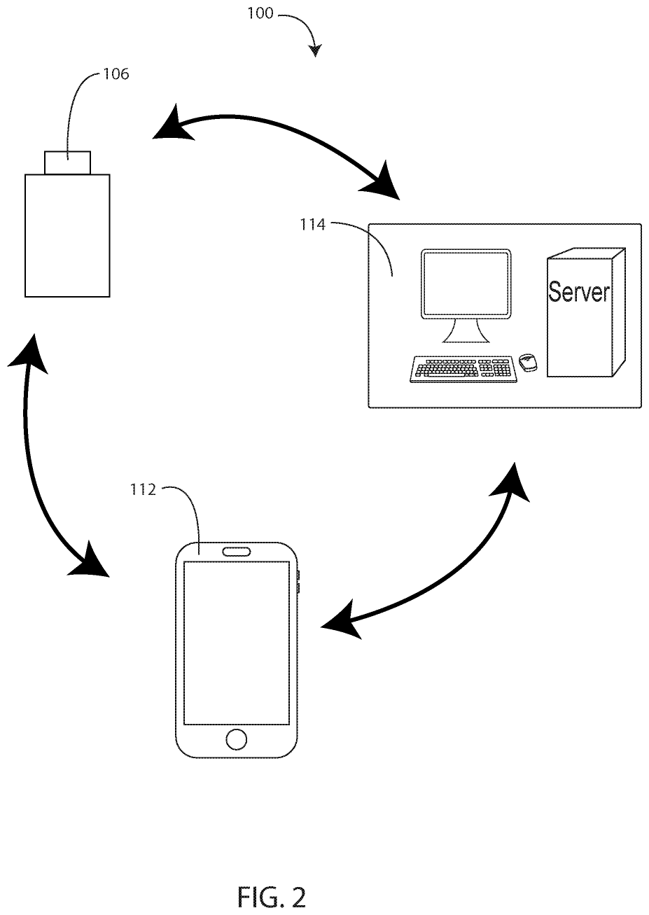

### Intoxication Monitoring System (FIGS. 1-2)

Referring now to FIG. 1, a schematic view of an intoxication monitoring system is shown in accordance with various embodiments herein. In various embodiments, the intoxication monitoring system 100 can include an ignition interlock system 102, a mobile device 112, and a server 114.

In various embodiments, the ignition interlock system 102 can include a vehicle 104 having a vehicle battery 110 and a vehicle control module 108 installed within. The ignition interlock system 102 can include an intoxication monitoring device 106 configured to selectively enable vehicle operation based on a test result, such as a breath test of the driver or another type of test for impairment. In some examples, the intoxication monitoring device 106 is coupled to the vehicle control module 108. The intoxication monitoring device 106 may be configured to detect a number of intoxicants that inhibit a user's ability to operate the vehicle.

In some embodiments, the intoxication monitoring device 106 and the vehicle control module 108 can form a Breath Alcohol Ignition Interlock device (BAIID) configured to selectively enable vehicle operation based on a driver's intoxication state. While the ignition interlock system 102 of the illustrated embodiment is configured to detect alcohol, it will be appreciated that the systems and methods described herein can be used in conjunction with interlock devices that are configured to detect various intoxicants, e.g., tetrahydrocannabinol (THC), opioids, narcotics, etc.

Various intoxication monitoring devices are disclosed in PCT Publication WO/2025/050052, titled “REMOVABLE MOUTHPIECE FOR BREATH DETECTOR,” published on Mar. 6, 2025, which is hereby incorporated by reference in its entirety, U.S. Pat. No. 10,604,011, titled “NETWORK INTOXICATION VEHICLE IMMOBILIZATION,” issued on Mar. 31, 2020, which is hereby incorporated by reference in its entirety, U.S. Pat. No. 7,934,577, titled “IGNITION INTERLOCK BREATHALYZER” issued on May 3, 2011, which is hereby incorporated by reference in its entirety, and U.S. Pat. No. 8,957,771, titled “APPARATUS, SYSTEM, AND METHOD FOR IMPLEMENTING AND MONITORING BREATH ALCOHOL TESTING PROGRAMS, USUALLY FROM A FIXED POINT LOCATION, SUCH AS A HOME” issued on Feb. 17, 2015, which is hereby incorporated by reference in its entirety.

In various embodiments, the vehicle control module 108 is configured to fit within a passenger compartment of a vehicle 104, such as under or behind the dashboard of the vehicle 104, within the center console of the vehicle, or both. In some embodiments, the intoxication monitoring device 106, the vehicle control module 108, or both can be configured to send and/or receive data, such as to other computing devices. In some embodiments, the intoxication monitoring device 106, the vehicle control module 108, or both can have wireless communication abilities. In some embodiments, the intoxication monitoring device 106, the vehicle control module 108, or both can include a cellular modem to facilitate wireless communication.

The intoxication monitoring device 106 can be coupled to the vehicle control module 108 via a wired connection. In an alternate embodiment, the connection between the vehicle control module 108 and the intoxication monitoring device 106 is wireless, such as using radio-frequency communications. In various embodiments, the installation has only the intoxication monitoring device 106 exposed in the vehicle for passenger interaction, which reduces the chance of tampering with ignition interlock system 102. The vehicle control module 108 may also be connected to other vehicle systems, such as to a power source, and/or to the ignition or other systems to monitor and/or control the operating state of the vehicle.

In various embodiments, the intoxication monitoring system 100 can include a mobile device 112. In various embodiments, the mobile device 112 can be a smartphone. Alternatively, the mobile device can be any of a computer tablet, laptop computer, cellular telephone, personal digital assistant, personal computer, smart watch, home monitoring device, and combinations thereof, or the like.

In various embodiments, the mobile device 112 is configured to interact with and provide information regarding the ignition interlock system 102. In some embodiments, the mobile device 112 can be configured to prompt the user to take a test with the intoxication monitoring device 106. In some embodiments, the mobile device 112 can be configured to provide the user with information such as the status of the ignition interlock system 102 (e.g., if calibration of the intoxication monitoring device 106 is required), the status of the user's account (e.g., if the user is up to date on payments), or the like. In some embodiments, the mobile device 112 can be configured to provide the user with feedback on how they operate the ignition interlock system 102 (e.g., if the user is using proper technique in providing the sample to the intoxication monitoring device 106).

In some embodiments, the intoxication monitoring device 106 can be in direct wireless communication with the control module 108. In some embodiments, the intoxication monitoring device 106 can be in wireless communication with the mobile device 112 and the mobile device can be in wireless communication with the control module 108, such that the intoxication monitoring device 106 is indirectly in communication with the control module.

In various embodiments, data and/or signals can be exchanged directly between the ignition interlock system 102 and the mobile device 112. Data and/or signals can be exchanged wirelessly using various techniques including inductive techniques (such as near-field magnetic induction-NFMI), 900 MHz communications, 2.4 GHz communications, communications at another frequency, FM, AM, SSB, BLUETOOTH™, Low Energy BLUETOOTH™, Long Range BLUETOOTH™, IEEE 202.11(wireless LANs) Wi-Fi, 202.15(WPANs), 202.16(WiMAX), 202.20, and cellular protocols including, but not limited to CDMA and GSM, ZigBee, and ultra-wideband (UWB) technologies.

In various embodiments, the intoxication monitoring system 100 can include a server 114, such as a server or a computing device. As will be described herein, the server 114 can include memory for storing data, a processor for processing data, and a network connection to send and receive data across a network with other portions of the intoxication monitoring system 100. In various embodiments, signals from various components of the system and/or notifications can be conveyed remotely such as to and/or through the cloud. The mobile device 112 may communicate with a server 114 over a network. The server 114 may process and store user data, calculate points and status levels, and manage rewards.

Referring now to FIG. 2, a schematic view of an intoxication monitoring system is shown in accordance with various embodiments herein. In various embodiments, the intoxication monitoring system 100 can include an intoxication monitoring device 106, a mobile device 112, and a server 114. The intoxication monitoring system 100 shown and described by FIG. 2 can function similarly to the intoxication monitoring system of FIG. 1, except the intoxication monitoring device 106 is a standalone device rather than being incorporated into an ignition interlock system 102. An intoxication monitoring system 100 having a stand-alone intoxication monitoring device 106 may be used in a number contexts, such as voluntary intoxication monitoring, situations in which an offender chooses not to drive, or the like.

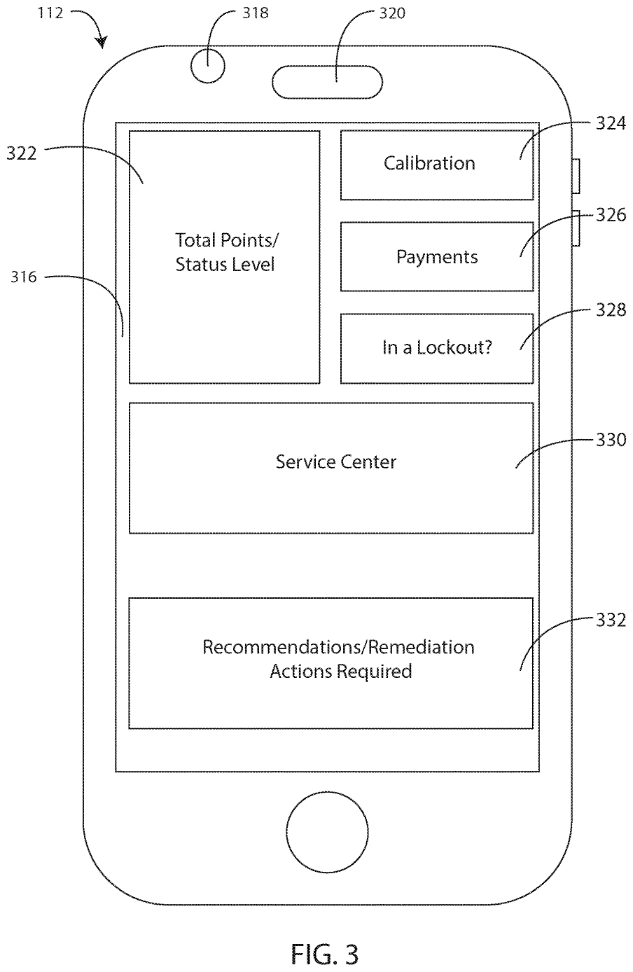

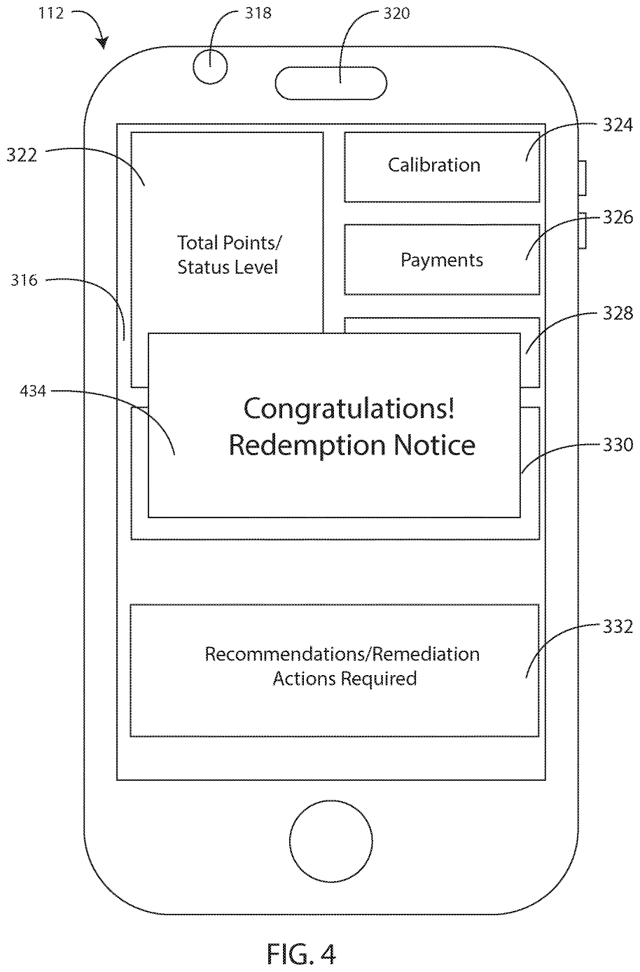

### Rewards System and User Interface (FIGS. 3-4)

It is desirable for a user intoxication monitoring system 100 to properly use and maintain the intoxication monitoring device 106 and equipment associated therewith (e.g., the vehicle battery 110). In various embodiments, the intoxication monitoring system 100 is configured to incentivize the user for proper use and maintenance of the intoxication monitoring system.

In various embodiments, the intoxication monitoring system 100 can include non-transitory computer-readable instructions executable by a processor. In various embodiments, the non-transitory computer-readable instructions can be stored in a memory of any of the intoxication monitoring device 106, mobile device 112, and server 114, or a combination of.

In various embodiments, the non-transitory computer-readable instructions include receiving data regarding the user's actions. In various embodiments, the non-transitory computer-readable instructions include analyzing the user action by applying a reward matrix criteria. In various embodiments, the non-transitory computer-readable instructions include changing a point total associated with the user's account when the user's action meets the reward matrix criteria. In various embodiments, the non-transitory computer-readable instructions include displaying, on a user interface, one or more redemption notifications selectable by the user when the point total surpasses a redemption threshold. In various embodiments, the non-transitory computer-readable instructions include receiving the user's redemption selection. In various embodiments, the non-transitory computer-readable instructions include processing the redemption selection.

Referring now to FIG. 3, a schematic view of a user interface is shown in accordance with various embodiments herein. In various embodiments, the mobile device 112 can include a user interface 316. In some embodiments, the mobile device 112 can also include a camera 318 and a speaker 320. The mobile device 112 can further include a processor, and a memory coupled to the processor. The memory may store non-transitory computer-readable instructions that, when executed by the processor, cause the mobile device to perform various methods and functions related to the intoxication monitoring system.

The mobile device 112 can be configured to generate one or more prompts to a user of the intoxication monitoring system 100 visually, audibly, and/or haptically. In various embodiments, the mobile device 112 can be configured to convey a prompt for a device wearer visually on user interface 316. In various embodiments, the prompt is provided audibly through the speaker 320. In various embodiments, the prompt is provided haptically through vibrations of the mobile device 112. In some embodiments, the prompt is provided through a separate external accessory device (such as an operably-connected smart watch, or the like).

The mobile device 112 can also be configured to receive input from a user of the intoxication monitoring system 100 audibly, through physical interaction, or by the use of gestures. In various embodiments, the mobile device 112 receives the input as a tactile input. For example, the user interface 316 of the mobile device 112 can be a touch screen. In various embodiments, the tactile input can include a tap, swipe, a button press, or the like.

In the example of FIG. 3, the user interface 316 of the mobile device 112 displays a home page for an intoxication monitoring system application. In various embodiments, the home page can include a plurality of icons. In various embodiments, when the user interacts with one of plurality of icons (e.g., by tapping the user interface 316), the user is directed to a corresponding rendering generated by the mobile device 112. The homepage depicted by FIG. 3 is for exemplary purposes only and the mobile device 112 may display any other suitable combination of icons.

In various embodiments, the home page can include an icon 322 for total points or status level. In various embodiments, upon interacting with the icon 322, the user is directed towards a page showing information regarding the total number of points associated with the user's account and/or the user's current status level.

In various embodiments, the home page can include an icon 324 for calibration. In various embodiments, upon interacting with the icon 324, the user is directed towards a page showing information regarding the calibration status of the intoxication monitoring device 106. Such information can include a re-calibration due date for the intoxication monitoring device 106, information regarding near-by calibration facilities, or the like.

In various embodiments, the home page can include an icon 326 for payments. In various embodiments, upon interacting with the icon 326, the user is directed towards a page showing information regarding payments. In some embodiments, the intoxication monitoring system 100 may charge a user a regular fee for continued use. In some embodiments, the intoxication monitoring system 100 may charge the user additional fees for lock out resets, device calibration, or the like. In various embodiments the payment page can include any of information regarding fees, an option for payment of a balance due, options for selecting method of payment, options for setting up automatic payment, or the like.

In various embodiments, the home page can include an icon 328 for lockouts. In various embodiments, upon interacting with the icon 328, the user is directed towards a page showing information regarding lockouts. In some embodiments, if a user provides a sample that exceeds an allowable intoxicant limit, the intoxication monitoring system 100 will enter a lockout state in which the vehicle 104 cannot be driven. In various embodiments, the icon 322 can direct the user towards information regarding vehicle lockout, options for requesting a lockout reset, or the like.

In various embodiments, the home page can include an icon 330 for service centers. In various embodiments, the icon 330 can display information regarding an established service center. The service center can be a service center assigned to the user or a service center close to the user's location as automatically determined using the mobile device 112. In various embodiments, upon interacting with the icon 330, the user is directed towards a page showing any of directions to the assigned service center, a list of near-by service centers, or the like.

In various embodiments, the home page can include an icon 332 for recommendations and remediation actions. In various embodiments, upon interacting with the icon 332, the user is directed towards a page containing a list of actions a user can take to increase their total points, increase their status level, avoid penalties, or the like.

Referring now to FIG. 4, a schematic view of a user interface is shown in accordance with various embodiments herein. In the example of FIG. 4, the user interface 316 of the mobile device 112 is displaying the home page for an intoxication monitoring system 100 as depicted by FIG. 3. In various embodiments, the user interface 316 of the mobile device 112 can further include a redemption notification 434.

In the example of FIG. 4, the redemption notification 434 is a visual icon that can appear on the user interface 316 of the mobile device 112 when the point total surpasses a redemption threshold. In various embodiments, upon interacting with the redemption notification 434, the user is directed towards a page showing one or more redemption options selectable by the user. The redemption options enable the user to exchange the points associated with their account for rewards.

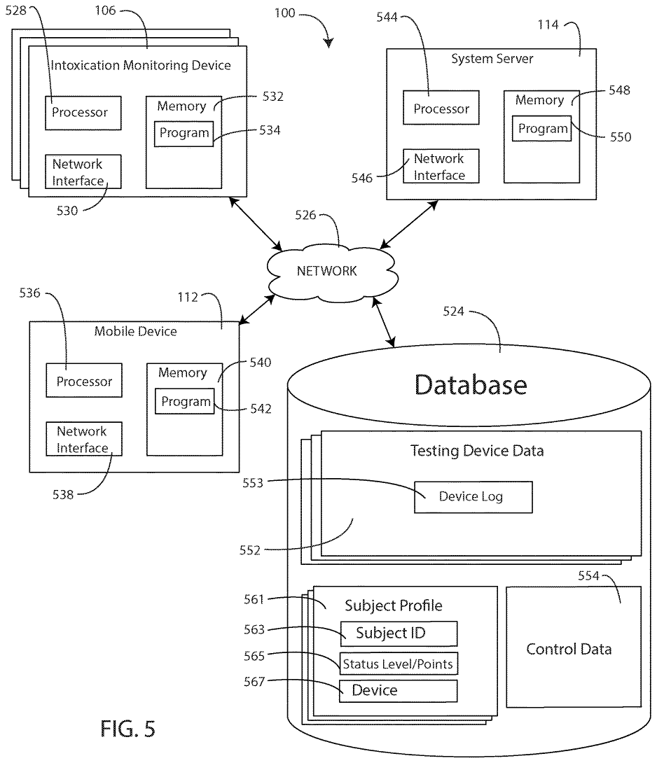

### System Component Overview (FIG. 5)

Referring now to FIG. 5, a schematic view of various components of an intoxication monitoring system in accordance with various embodiments herein. FIG. 5 shows a schematic of the intoxication monitoring system 100 that can be used to analyze and incentivize a user's actions. The intoxication monitoring system 100 can also receive, store, process, transfer, and present data related to intoxicant monitoring of various subjects. The data recorded by the intoxication monitoring device 106 can be managed in part by a server 114 and stored in a database 524, such as a testing database within the database 524.

The intoxication monitoring system 100 shown in FIG. 5 can include an intoxication monitoring device 106, a mobile device 112, and a server 114. These devices 106, 112, 114 can be computer systems or include computer systems. In various embodiments, these computing systems can each include a processor 528, 536, 544, memory and/or storage 532, 540, 548 and a network interface 530, 538, 546 to allow communications over network 526. The memory 532, 540, 548 is shown in FIG. 5 as including programming 534, 542, 550 that can control the processors 528, 536, 544. It should be understood that other devices that include computing systems can also include similar elements, such as a processor, network interface, memory, and computer programming.

In some embodiments, the server 114 can take the form of or include a standard computer system, or, in some embodiments, can take the form of or include a mobile device, such as a smartphone or tablet computer. In some embodiments, the server 114 can be implemented using a plurality of separate, individual computers.

A standard computer system can operate application software or browser software, such as programs 534, 542, 550, which can be stored in memory 532, 540, 548 on the respective devices. The programs can allow the devices to communicate over the network 526 as part of the intoxication monitoring system 100.

In particular, the programming 534, 542 can allow communication with the server 114 over the network 526. In some embodiments, the intoxication monitoring system 100 will be designed to allow direct communication between the mobile device 112 and the intoxication monitoring device 106.

In various embodiments, data from an intoxication monitoring device 106 such as testing data or test events can be stored in the database 524. The server 114 can be in communication with a plurality of other intoxication monitoring devices and may aggregate all their data in a single database 524. In other embodiments, data from separate intoxication monitoring devices can remain separated into separate databases 524 that are still accessible to the server 114.

The computers of devices 106, 112, 114 can be a computing device that includes a processor for processing computer programming instructions. In most cases, the processor can be a CPU. Various different CPU devices are possible, such as those created by Intel Corporation (Santa Clara, California), Advanced Micro Devices, Inc (Santa Clara, California), or a RISC processor produced according to the designs of Arm Holdings PLC (Cambridge, England). Additionally, the computers can include memory. The memory can be in the form of both temporary, random access memory (RAM) and more permanent storage such a magnetic disk storage, FLASH memory, or another non-transitory (also referred to as permanent) storage medium. The memory and storage, which can be referred to collectively as “memory”, can contain both programming instructions and data. In various embodiments, both programming and data can be stored permanently on non-transitory storage devices and transferred into RAM when needed, such as for processing or analysis. In some embodiments, the computers can include a graphics processing unit (or GPU) for processing of visual input and outputs.

In various embodiments, the various data can be stored locally and/or stored remotely. In FIG. 5, the data can be stored in database 524. In many embodiments, the database 524 can be a portion of the server 114. In other embodiments, the database 524 can be a separate element from the server 114. In some embodiments, the database 524 can consist of local storage on the various devices included in the intoxication monitoring system 100.

In various embodiments, the database 524 can include defined database entities that, in some embodiments, can constitute database tables in a relational database. In other embodiments, entities may represent database objects or any other type of database entity usable with a computerized database. In various embodiments, the phrase database entity can refer to data records in a database, which can include a row in a database table, an instantiation of a database object, or any other populated database entity. Data within this database 524 can be “associated” or “linked” with other data. This association can be implemented in a variety of techniques depending on the technology used to store and manage the database, such as through formal relationships in a relational database or through established relationships between objects in an object-oriented database.

Database 524 can include intoxication monitoring device data 552. Intoxication monitoring device data 552 can be created by the intoxication monitoring device 106 in response to a testing event. The test event data can be received from the intoxication monitoring device 106. In various embodiments, the intoxication monitoring device 106 can create test event data for each test event. In some embodiments, the test event data is stored within the subject profile 561 for the subject that is associated with the test event data. In some embodiments, the user profile is linked with the test event data within the testing database, such as to identify which subject the test event data is associated with. As will be described herein, the database may receive intoxication monitoring device data 552 through one or more device logs 553 generated by the intoxication monitoring device 106.

Database 524 can include control data 554, such as one for each intoxication monitoring device 106. The control data 554 can be intoxication monitoring device 106 specific. In various embodiments, the concentration limits for different substances can be different. As an example, in some embodiments, the alcohol concentration limit can be set as 0.02%, whereas a tetrahydrocannabinol (THC) concentration limit can be set as 0, such that any detectable amount can result in a violation or the inability to start a vehicle. Alternatively, both an alcohol concentration limit and a THC concentration limit can have a non-zero value.

In various embodiments, the database 524 can further include a plurality of subject profiles 561, such as one subject profile for each subject that has been issued a subject testing device. The subject profile 561 can include a subject identifier 563. The subject identifier 563 can include information for identifying the subject or other personal information, such as the subject's name, address, email address, phone number, birthdate, driver's license number, identification number, or the jurisdiction that issued his/her driver's license. The subject profile 561 can further include a user status level and/or points 565. The subject profile 561 can also include compliance device data 567, such as data identifying the type of subject testing device and/or an identification number of the subject testing device that has been issued to the subject. The subject profile 561 can further include compliance information, such as when the subject was issued the compliance device, rental or lease information regarding the compliance device, or payment/fee status. Intoxication monitoring system 100 can include the transfer and receival of various portions of data between the various devices and components of the intoxication monitoring system 100.

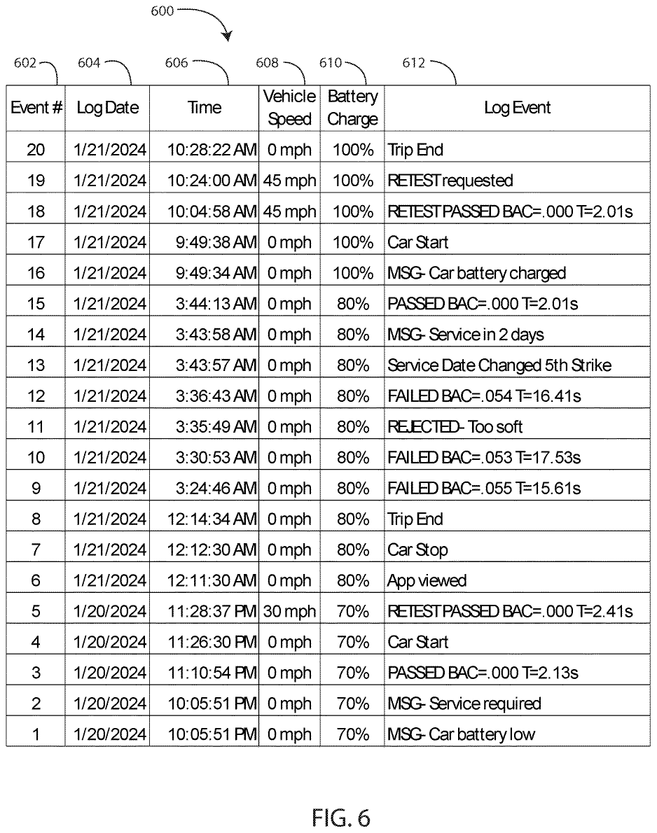

### Device Logs (FIGS. 6-8)

In various embodiments, the intoxication monitoring system 100 is configured to generate a plurality of device logs. As used herein, the term “device log” refers to a recorded data structure encompassing a series of log entries that document discrete log events occurring within an intoxication monitoring system. In various embodiments, the device logs can be generated at the intoxication monitoring device 106, the mobile device 112, or combinations thereof. In various embodiments, one or more device logs can be conveyed from the intoxication monitoring device 106 to the mobile device 112 and/or the server 114. In various embodiments, each device log is stored within the system's database 524, such as within the testing device data 552.

In various embodiments, each time a log event occurs, a log entry is generated. As used herein, the term “log event” refers to a discrete occurrence or action detected within an intoxication monitoring system that is recorded as an entry in a device log. A log event may represent a user interaction with the intoxication monitoring system, a system status change, or other relevant activity captured by components of the intoxication monitoring system (e.g., the intoxication monitoring device 106 or the mobile device 112).

In various embodiments, a device log can contain one more log entries. In various embodiments, a device log can take the form of a table that can be accessible to authorized monitoring entities via established network protocols and security measures for oversight and analysis. Such monitoring authorities may include any of law enforcement, correctional authorities, civil organizations (e.g., a trucking company that monitors the drivers of its fleet of trucks), intoxication monitoring system companies, service centers, or the like.

FIGS. 6-8 illustrate a plurality of exemplary device logs in accordance with various embodiments herein. The device logs illustrated by FIGS. 6-8 are shown in a tabular format. However, it should be noted that any other suitable means of displaying information is possible. In various embodiments, a device log depicts a series of log entries over a particular time frame. The particular time frame can be any suitable duration of time such as a day, two days, a week, a month, or the like.

Referring now to FIG. 6, a schematic view of a device log is shown in accordance with various embodiments herein. FIG. 6 depicts a device log 600 in a tabular form showing data generated by a user of the intoxication monitoring system 100 over a particular time frame.

In various embodiments, a log entry is generated each time a log event occurs over the particular time frame. Each log entry can be entered as a row in the device log 600. In the example of FIG. 6, the device log contains twenty rows corresponding to twenty log entries. However, a device log may have greater or fewer than 20 rows depending on the number of log event that occur over the particular time frame.

In various embodiments, multiple types of data can be transferred to the server 114 along with each long entry. Such data can include any of vehicular speed, location of the intoxication monitoring device 106, mobile device 112, and/or vehicle 104, vehicle battery voltage, intoxication test results, images of the user and/or surrounding environment, or the like.

In various embodiments, the device log can contain a plurality of columns with each column containing a piece of information relevant to the log entries. In the example of FIG. 6, the device log contains six columns. It should be noted that device logs can contain any suitable number of columns in any suitable order, depending on how many pieces of information are displayed with each log entry. In various embodiments, a device log may include two, three four, five, six, or more columns.

In various embodiments, the device log 600 can include a column 602 to label each log entry in the device log. In the example of FIG. 6, column 602 is titled “Event #” and each entry under column 602 is a number labeling the associated log entry. For instance, the first log entry in the device log 600 is labeled “1” and the last log entry in the device log 600 is labeled “20.” In alterative embodiments, the log entries can be labeled according to any other suitable scheme such as A to Z, or the like.

In various embodiments, the device log 600 can include a column 604 to display a date associated with each log entry in the device log. In various embodiments, the date displayed in column 604 corresponds to the date that that the log event occurred. In the example of FIG. 6, column 604 is titled “LogDate” and each entry under column 604 is a date (displayed as Month/Day/Year) associated with the log entry. For instance, the first log entry in the device log 600 has an associated date of Jan. 20, 2024 and the last log entry in the device log 600 has an associated date of Jan. 21, 2024. In alterative embodiments, the log entries can be labeled according to any other convention of writing dates.

In various embodiments, the device log 600 can include a column 606 to display a time associated with each log entry in the device log. In various embodiments, the time displayed in column 606 corresponds to the time that the log event occurred. In the example of FIG. 6, column 606 is titled “Time” and each entry under column 606 is a time (displayed as Hours: Minutes: Seconds AM/PM) associated with log entry. For instance, the first log entry in the device log 600 has an associated time of 10:05:51 PM and the last log entry in the device log 600 has an associated time of Oct. 28, 2022 PM. In alterative embodiments, the log entries can be labeled according to any other convention of writing times.

In various embodiments, the device log 600 can include a column 608 to display a vehicle speed associated with each log entry in the device log. In various embodiments, the vehicle speed displayed in column 608 corresponds the speed that the vehicle 104 was travelling when the corresponding log event occurred. In the example of FIG. 6, column 608 is titled “Vehicle Speed” and each entry under column 608 is a speed (in miles per hour) associated with log entry. For instance, the first log entry in the device log 600 has an associated speed of 0 mph and the last log entry in the device log 600 has an associated speed of 0 mph. In alterative embodiments, vehicle speed can be expressed in any other suitable units such as kilometers per hour, or the like.

In various embodiments, the device log 600 can include a column 610 to display a battery charge associated with each log entry in the device log. In various embodiments, the battery charge displayed in column 608 corresponds the level of charge that the vehicle battery 110 was at when the corresponding log event occurred. In some embodiments, the ignition interlock system 102 is configured to determine the charge of the vehicle battery 110 only when a trip is not in progress. In some embodiments, the ignition interlock system 102 can be operably connected to the vehicle's control system and is additionally configured to determine the charge of the vehicle battery 110 when a trip is in progress. In the example of FIG. 6, column 610 is titled “Battery Level” and each entry under column 610 is a vehicle battery level expressed as a percentage (with 100% corresponding to a completely full vehicle battery and 0% corresponding to a completely empty vehicle battery) associated with log entry. For instance, the first log entry in the device log 600 has an associated vehicle battery level of 70% of and the last log entry in the device log 600 has an associated vehicle battery level of 100%. In alterative embodiments, the log entries can be labeled according to any other convention expressing battery charge level.

In various embodiments, the device log 600 can include a column 612 to display the log event that is associated with each log entry in the device log. In the example of FIG. 6, column 612 is titled “LogEvent” and each entry under column 612 is one of a plurality of log events detected by the ignition interlock system.

In various embodiments, each time a log event occurs, a log entry is generated. Log events can include particular actions taken by the user the intoxication monitoring system 100, components of intoxications monitoring system having a particular status, or the like. In various embodiments, the log events in column 612 of device log 600 can include instances of the vehicle 104 starting up or shutting off (see for example log events 4, 7, and 17 with event label “Car Start”). In various embodiments, log events in device log 600 can include instance of the user taking an intoxication test (see for example log event 15 with event label “PASSED BAC=0.000 T=2.01s”). In various embodiments, log events in device log 600 can include instances of the system reminding the user to service the intoxication monitoring device (see for example log event 2 with event label “MSG-Service required”).

The device log 600 shows multiple types of events. In alterative embodiments, the intoxication monitoring system 100 is configured to divide the log events by type and to generate multiple device logs with each device log containing log events having a particular type or types.

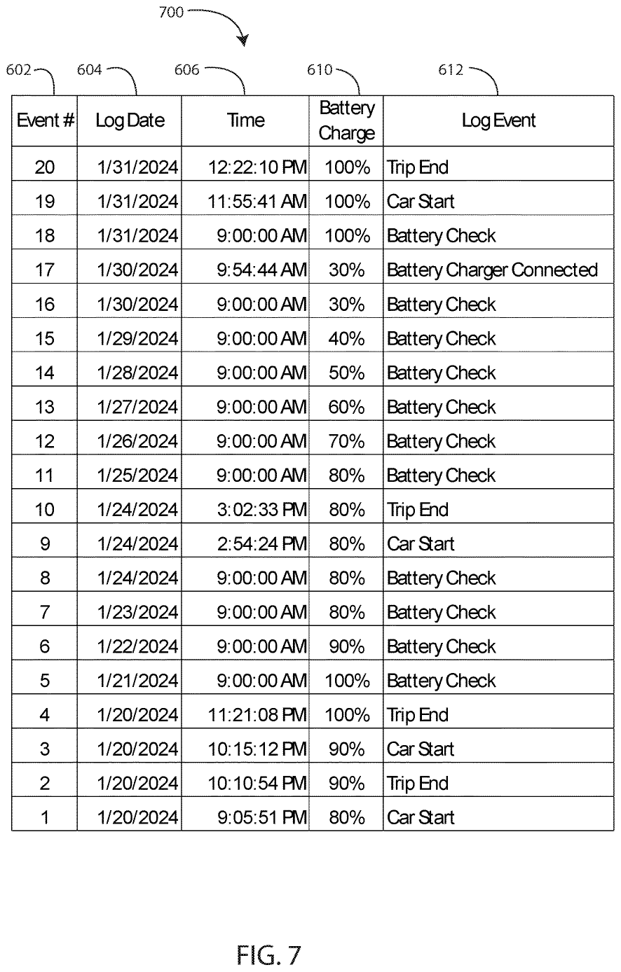

Referring now to FIG. 7, a schematic view of a device log is shown in accordance with various embodiments herein. In various embodiments, the device log 700 depicts a subset of the types of log entries that are displayed in the device log 600. The device log 700 is configured to display only the log entries that are relevant to the power state of the vehicle 104 and the level of charge of the vehicle battery 110.

FIG. 7 depicts a device log 700 in a tabular form showing log events over a particular time frame. Like the device log 600, the device log 700 contains columns 602, 604, 606, 610, and 612. Notably that column 608 is absent from the device log 700 because vehicle speed is not relevant to the log entries for device log 700.

Device log 700 contains a subset of log event that pertain to the state of the vehicle battery 110. In various embodiments, an ignition interlock system 102 utilizes the vehicle battery 110 by establishing an electrical connection that facilitates the operation of system components. The vehicle battery 110 can serve as a primary power source, ensuring that essential monitoring functions are operational during both vehicle use and while the vehicle is stationary. Accordingly, it is important for the user of an ignition interlock system to maintain the vehicle battery at a sufficiently high charge.

In various embodiments, when the vehicle 104 is in operation, the alternator can supply electrical energy to recharge the vehicle battery 110, thus sustaining the necessary power levels for the ignition interlock system 102. However, in situations where the vehicle 104 is not frequently used, the vehicle battery 110 can deplete, necessitating alternative charging mechanisms. Accordingly, it is beneficial for the device log 700 to track how frequently the vehicle's alternator is turned on. In the example of FIG. 7, the device log tracks instances of the vehicle 104 being started. For instances log event numbers 1, 3, 9, and 19 all log instances of a “car start.”

In various embodiments, the intoxication monitoring system is configured to periodically check the vehicle batter level. In the example of FIG. 7, the device log displays instances of a daily vehicle battery level check. For instances log event numbers 5-8, 11-16, and 18 all log instances of a “Battery Check.”

In various embodiments, the intoxication monitoring system is configured to detect when a charger has been connected to the vehicle battery 110. In the example of FIG. 7, log event numbers 17 logs an instance of a “Battery Charger connected.”

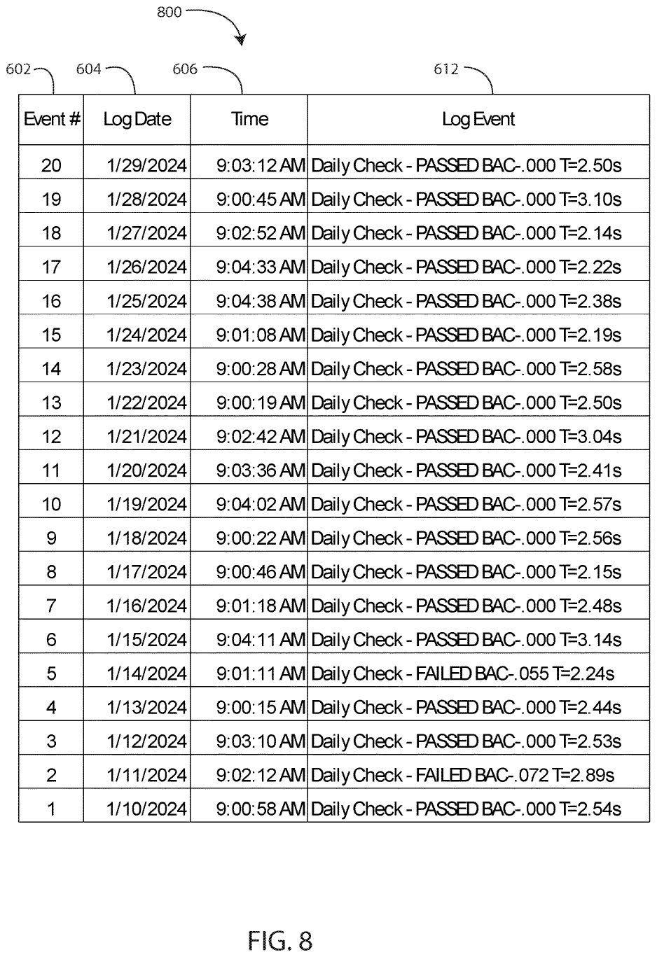

Referring now to FIG. 8, a schematic view of a device log is shown in accordance with various embodiments herein. In various embodiments, the device log 800 depicts a subset of the types of log entries that are displayed in the device log 600. The device log 800 is configured to display only the log entries that are relevant to intoxication test results of the user. In various embodiments, each time the user takes a test using intoxication monitoring device 106, a log event is generated.

FIG. 8 depicts a device log 800 in a tabular form showing log events over a particular time frame. Like the device log 600, the device log 700 contains columns 602, 604, 606, and 612. Notably that columns 608 and 610 are absent from the device log 700 because vehicle speed and battery level are not relevant to the log entries for device log 800.

Device log 800 is configured to draw attention to the intoxication test results of the user. In various embodiments, the intoxication monitoring system can prompt the user to perform a daily intoxication test. The device log 700 shows the results of a user's daily intoxication test from the dates Jan. 10, 2024 to Jan. 29, 2024. Each log event in device log 700 contains data pertaining to a daily intoxication test result. In the example of FIG. 7, each log entry displayed column 612 depicts whether the user passed the intoxication test, the BAC detected by the intoxication monitoring device 106, and the duration of the breath sample provided to the intoxication monitoring device. Alternatively, the data displayed in column 612 can vary depending on a number of factors such as the type of intoxication test administered (e.g., transdermal vs breath), the type of substance being tested for (e.g., alcohol vs. THC), or the like.

The Device log 700 shows several instances of passed intoxication tests. For instance, log entry 1 shows passed intoxication test with the user providing a sample having a BAC of 0.000 and a breath duration of 2.54 seconds. The Device log 700 shows several instances of failed intoxication tests. For instance, log entry 2 shows failed intoxication test with the user providing a sample having a BAC of 0.072 and a breath duration of 2.89 seconds.

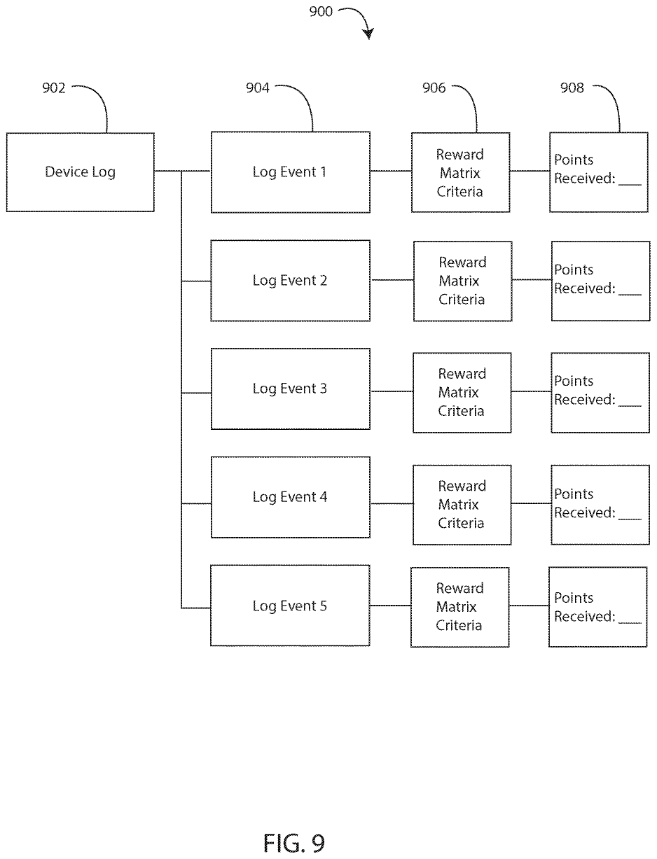

### Device Log Rewards Matrix (FIGS. 9-13)

In various embodiments, the intoxication monitoring system 100 is configured to apply gamification principles to enhance user compliance and engagement by reinforcing positive behavior through a structured rewards framework. The intoxication monitoring system 100 can integrate a reward matrix to systematically assesses user actions based on predefined criteria. The criteria encapsulate various aspects of proper device usage and maintenance, including compliance with testing protocols, vehicle battery maintenance, and adherence to scheduled calibrations.

In various embodiments, the intoxication monitoring device 106 (which in some circumstances can be incorporated into an ignition interlock system 102) is configured to receive data regarding a user's actions. Such data may include, but is not limited to, test results, vehicle operation metrics, and interaction metrics. The intoxication monitoring device 106 can then transmit the data to one or more of the mobile device 112 or the server 114.

In various embodiments, the intoxication monitoring system 100 is then configured to analyze each user action by applying a reward matrix criteria. Such criteria may determine whether specific user actions contribute positively to the goal of device compliance and sobriety adherence. In some embodiments, the server 114 performs the analysis at processor 544. In some embodiments, the mobile device 112 performs the analysis at processor 536. In some embodiments, the intoxication monitoring system 100 is configured to analyze user actions through the review of one or more device logs.

Referring now to FIG. 9, a processing scheme for analyzing device logs is shown in accordance with various embodiments herein. In various embodiments the processing scheme 900 can be executed in open or more processors of the mobile device 112 and/or the server 114. In various embodiments, the processing scheme 900 is configured to analyze device log 902. Device log 902 can be a device log generated at the intoxication monitoring device 106 such as device logs 600, 700, and 800 shown and described in FIGS. 6-8.

In various embodiments, the processing scheme is configured to divide the device log into discrete log events 904. As described above, each log event is a discrete occurrence or action detected within an intoxication monitoring system that is recorded as a log entry in the device log. A log event may represent a user interaction, system status change, or other relevant activity captured by components within the system, such as an ignition interlock device or associated mobile device. Each log entry associated with a log event typically includes data such as the time of occurrence, nature of the event, and any pertinent parameters like vehicular speed or test results, allowing remote monitoring entities to review and assess system performance and compliance.

In some embodiments, the processing scheme is further configured to categorize each log event 904 into one of a plurality of predefined categories. This categorization aids in organizing the disparate data types into logical groupings that reflect distinct user behaviors or system responses. Categories may include, for example, user compliance events, system calibration events, device power status events, user test results, or the like. This structured categorization allows the system to apply specific reward matrix criteria relevant to each category, thereby enabling a nuanced evaluation of user interactions.

In various embodiments, the processing scheme 900 is configured to apply a rewards matrix to the device log 902 by applying a rewards matrix criteria 906 to each log event 904. The rewards matrix can include a set of criteria aligned with desired user behaviors and system performance objectives and can encompass various performance metrics such as successful completion of required intoxication tests, regular maintenance of necessary vehicle battery levels, timely re-calibration of devices, and other factors that contribute to optimal system operation and reliability.

In various embodiments, the processing scheme 900 is configured to change a point total associated with the user's account when the user's action meets the rewards matrix criteria 906. Upon categorization of log events, the processing scheme 900 evaluates each log event 904 event against a rewards matrix criteria 906. Based on the evaluation the processing scheme 900 can selectively apply points 908 to the user's account. For instance, if a log event indicates that a user successfully completed a scheduled sobriety test with acceptable results, this may lead to an allocation of points or rewards to the user's account. Conversely, failure to meet scheduled maintenance requirements may result in no points being awarded.

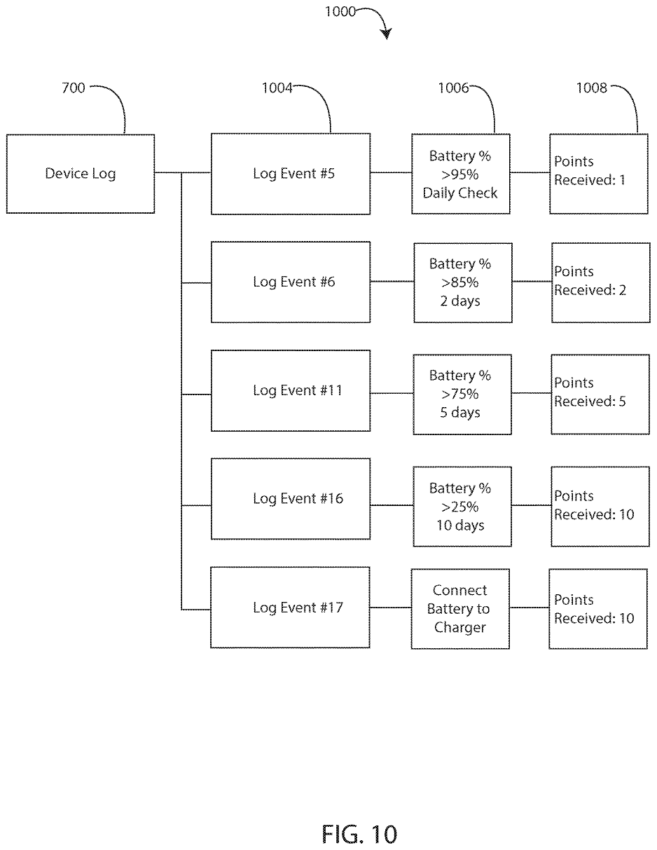

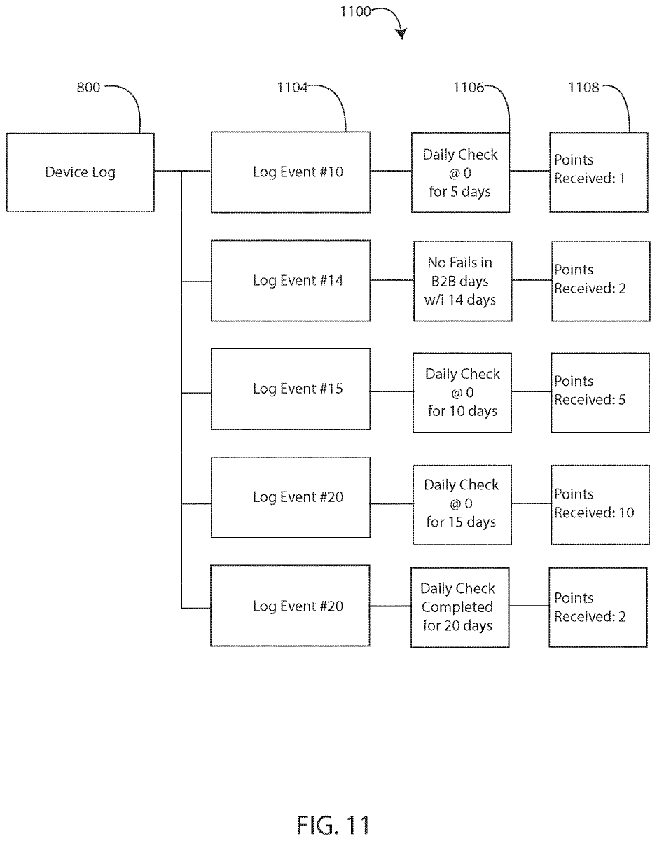

FIG. 9 provides an exemplary processing scheme 900 that applies a generic rewards matrix criteria 906 to a generic device log 902. Specific examples of how rewards matrices can be applied to a device log are depicted by FIGS. 10-11 herein.

Referring now to FIG. 10, a processing scheme for analyzing device logs is shown in accordance with various embodiments herein. In various embodiments the processing scheme can be executed in open or more processors of the mobile device 112 and/or the server 114.

In various embodiments, the processing scheme 1000 is configured to analyze one or more device logs. In the example of FIG. 10, the device log being analyzed by the processing scheme 1000 is device log 700 shown by FIG. 7. As described above, the device log 700 is configured to display only the log entries that are relevant to the power state of the vehicle 104 and the level of charge of the vehicle battery 110.

In various embodiments, the processing scheme 1000 is configured to apply a rewards matrix to the device log 700 by applying a rewards matrix criteria 1006 to each log event 1004. In various embodiments, the user's action is deemed compliant the user completes vehicle management tasks, such as ensuring the vehicle battery 110 maintains a suitable charge level over a defined period. The intoxication monitoring system 100 facilitates the process by actively tracking vehicle battery status and system interactions to collect pertinent data. A vehicle battery is considered to have a suitable level when conditions are met, such as operating the vehicle's engine for at least 15 minutes every other day, thereby ensuring sufficient charge is maintained. Alternatively, connecting the vehicle battery to an external charger further satisfies the criteria.

In the example of FIG. 10, the processing scheme 1000 is configured to apply rewards matrix criteria 1006 to several log events from device log 700. Log event 5 in device log 700 is a daily battery check. As can be seen in the device log 700 in FIG. 7, the vehicle battery level at the instance of log event 5 is 100%. A rewards matrix criteria 1006 that the vehicle battery level is above 95% at the daily battery level check is applied to log event 5. Since the vehicle battery level is at 100% at the instance of log event 5, the user is rewarded points 1008. In this instance, the user is rewarded one point. Conversely, if the vehicle battery level were below 95%, the user would not be rewarded any points.

Log event 6 in device log 700 is a daily battery check. As can be seen in the device log 700 in FIG. 7, the vehicle battery level at the instance of log event 6 is 90%. A rewards matrix criteria that the vehicle battery level is above 85% at the daily battery level check for at least two consecutive days is applied to log event 6. Since the vehicle battery level is at 90% at the instance of log event 6 and at 100% at log event 5 (the battery level from the previous day's daily battery level check), the user is rewarded points 1008. In this instance, the user is rewarded two points. Conversely, if the vehicle battery level were below 85% over the two consecutive days, the user would not be rewarded any points.

Log event 11 in device log 700 is a daily battery check. As can be seen in the device log 700 in FIG. 7, the vehicle battery level at the instance of log event 11 is at 80%. A rewards matrix criteria that the vehicle battery level is above 75% at the daily battery level check for at least five consecutive days is applied to log event 11. Since the vehicle battery level is at 80% at the instance of log event 11 and was above 75% at the previous four daily battery checks (log events 8, 7, 6, and 5), the user is rewarded points 1008. In this instance, the user is rewarded five points. Conversely, if the vehicle battery level dropped below 80% over the five consecutive days, the user would not be rewarded any points.

Log event 16 in device log 700 is a daily battery check. As can be seen in the device log 700 in FIG. 7, the vehicle battery level at the instance of log event 16 is 30%. A rewards matrix criteria that the vehicle battery level is above 25% at the daily battery level check for at least ten consecutive days is applied to log event 16. Since the vehicle battery level is at 30% at the instance of log event 16 and was above 25% at the previous nine daily battery checks (log events 15, 14, 13, 12, 11, 8, 7, 6, and 5), the user is rewarded points 1008. In this instance, the user is rewarded ten points. Conversely, if the vehicle battery level dropped below 30% over the ten consecutive days, the user would not be rewarded any points.

Log event 17 in device log 700 is an instance of the user connecting the vehicle battery to a charger. A rewards matrix criteria that the vehicle battery be connected to a charger is applied to log event 17. Since the vehicle battery was connected to a charger, the user is rewarded points 1008. In this instance, the user is rewarded ten points. Conversely, if the vehicle battery level were not connected to a charger, the user would not be rewarded any points.

Referring now to FIG. 11, a processing scheme for analyzing device logs is shown in accordance with various embodiments herein. In various embodiments the processing scheme 1100 can be executed in open or more processors of the mobile device 112 and/or the server 114.

In various embodiments, the processing scheme 1100 is configured to analyze device log. In the example of FIG. 11, the device log being analyzed by the processing scheme 1000 is device log 800 shown by FIG. 8. As described above, the device log 800 is configured to display only the log entries that are relevant to the intoxication test results of the user.

In various embodiments, the processing scheme 1100 is configured to apply a rewards matrix to the device log 800 by applying a reward matrix criteria 1106 to each log event 1104. In various embodiments, the intoxication monitoring system can prompt the user to perform a daily intoxication test. The rewards matrix in processing scheme 1100 can be configured to evaluate user actions based on predefined compliance thresholds, such as passing a daily intoxication test.

In the example of FIG. 11, the processing scheme 1100 is configured to apply reward matrix criteria 1108 to several log events from device log 800. Log event 10 in device log 800 is a daily intoxication check. As can be seen on the device log 800 in FIG. 8, the user has passed the daily intoxication check by providing a breath sample having a BAC of 0.000. A rewards matrix criteria that the user has passed the daily intoxication check with a BAC of 0.000 for five consecutive days is applied to log event 10. Since the user has provided a sample of 0.000 at log event 10 and for the past four consecutive days (log events 9, 6, 7, and 6), the user is rewarded points 1108. In this instance, the user is rewarded one point. Conversely, if the user provided a non-zero BAC breath sample over the past five consecutive days, the user would not be rewarded any points.

Log event 14 in device log 800 is a daily intoxication check. As can be seen on the device log 800 in FIG. 8, the user has passed the daily intoxication check by providing a breath sample having a BAC of 0.000. A rewards matrix criteria that the that the user has no back-to-back fails (fails for two consecutive days) of the daily intoxication check over the past 14 days is applied to log event 14. Since the user has only failed the intoxication test on nonconsecutive days (log events 2 and 5) within the past 14 days, the user is rewarded points 1108. In this instance, the user is rewarded two points. Conversely, if the user had back-to-back fails in the past fourteen days, the user would not be rewarded any points.

Log event 15 in device log 800 is a daily intoxication check. As can be seen on the device log 800 in FIG. 8, the user has passed the daily intoxication check by providing a breath sample having a BAC of 0.000. A rewards matrix criteria that the user has passed the daily intoxication check with a BAC of 0.000 for ten consecutive days is applied to log event 15. Since the user has provided a sample of 0.000 at log event 15 and for the past nine consecutive days (log events 14, 13, 12, 11, 10 9, 6, 7, and 6), the user is rewarded points 1108. In this instance, the user is rewarded five points. Conversely, if the user provided a non-zero BAC breath sample over the past ten consecutive days, the user would not be rewarded any points.

Log event 20 in device log 800 is a daily intoxication check. As can be seen on the device log 800 in FIG. 8, the user has passed the daily intoxication check by providing a breath sample having a BAC of 0.000. A rewards matrix criteria that the that the user has passed the daily intoxication check with a BAC of 0.000 for 15 consecutive days is applied to log event 20. Since the user has provided a sample of 0.000 at log event 20 and for the past fourteen consecutive days (log events 6-19), the user is rewarded points 1108. In this instance, the user is rewarded ten points. Conversely, if the user provided a non-zero BAC breath sample over the past fifteen consecutive days, the user would not be rewarded any points.

Log event 20 in device log 800 is a daily intoxication check. As can be seen on the device log 800 in FIG. 8, the user has passed the daily intoxication check by providing a breath sample having a BAC of 0.000. A rewards matrix criteria that the that the user has completed the daily intoxication check for 15 consecutive days is applied to log event 20. Since the user has completed the daily intoxication check at log event 20 and for the past nineteen consecutive days (log events 1-19), the user is rewarded points 1108. In this instance, the user is rewarded ten points. Conversely, if the user missed one or more daily intoxication checks over the twenty consecutive days, the user would not be rewarded any points.

In the context of applying the reward matrix criteria, the user actions undergo evaluation through a comparison mechanism where each user action is compared against predefined criteria established in the reward matrix. In various embodiments, applying the reward matrix criteria includes comparing elements of the reward matrix criteria with the user's actions to determine if the elements of the reward matrix criteria have been fulfilled. During this evaluation, user actions such as regular operational checks and adherence to device prompts serve as key indicators, allowing for a comprehensive assessment that determines the subsequent allocation of rewards based on the degree of criteria fulfillment.

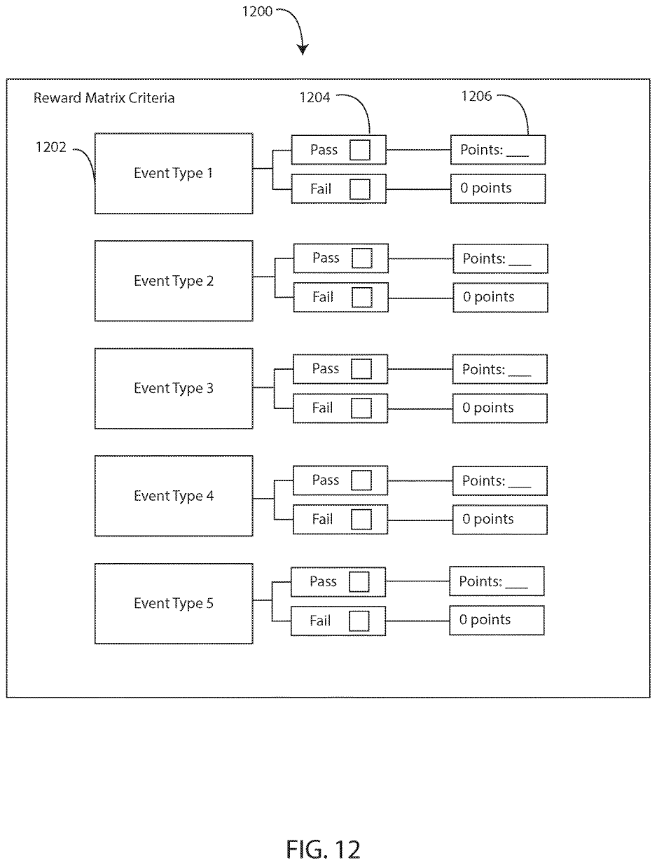

Referring now to FIG. 12, a schematic view of reward matrix criteria is shown in accordance with various embodiments herein. In various embodiments the rewards matrix criteria 1200 can include a plurality of event types 1202. In the example of FIG. 12, the rewards matrix criteria 1200 includes five event types. However, the rewards matrix criteria 1200 can include any suitable number of event types such as three, four, six, or more event types.

In various embodiments, each event type 1202 can include a certain event criteria 1204 that a user must meet. If the user meets the event criteria 1204 the user receives a “Pass” and is rewarded a predetermined number of points 1206. If the user does not meet the event criteria the user receives a “Fail” and is rewarded zero points.

In various embodiments the rewards matrix criteria 1200 can include event types 1202 pertinent to vehicle battery level. Such event types can include events that incentivize the user to maintain the vehicle battery level. In some embodiments, the user's action meets the reward matrix criteria 1204 if the user's action includes maintaining a suitable vehicle battery level for a specific amount of time. The threshold for a suitable battery level for meeting the rewards matrix criteria can be set by the intoxication monitoring system 100. In some embodiments, the user's action meets the reward matrix criteria if the vehicle battery 110 has a charge of greater than or equal to 20 %, 30 %, 40 %, 50 %, 60 %, 70 %, 80 %, 90 %, or 100 %, or can be an amount falling within a range between any of the foregoing over the specific amount of time. The duration of the specific amount of time can be set by the intoxication monitoring system 100. In some embodiments, the user's action meets the reward matrix criteria if they maintain the suitable battery level for a duration greater than or equal to 1 days, 6 days, 11 days, 16 days, 21 days, 26 days, or 30 days, or can be an amount falling within a range between any of the foregoing.

In some embodiments, the user's action meets the reward matrix criteria if the user's action comprises running the vehicle for a predetermined duration and/or at a predetermined frequency. In one example, the user's action meets the reward matrix criteria if the vehicle runs for at least 15 minutes every other day. Alternatively, rather than running the vehicle, the user can connect the vehicle battery 110 to a charger. In one example, the user's action meets the reward matrix criteria if the vehicle runs the vehicle 104 for at least 15 minutes every other day or if the vehicle battery 110 is attached to a charger.

Further, the predetermined duration and predetermined frequency for charging the battery (either by running the vehicle 104 or connecting a charger) can be set by the intoxication system. In some embodiments, the predetermined duration for running the vehicle or connecting the charger can be greater than or equal to 5 minutes, 8 minutes, 12 minutes, or 15 minutes. In some embodiments, the predetermined duration can be less than or equal to 60 minutes, 45 minutes, 30 minutes, or 15 minutes. In some embodiments, the predetermined duration can fall within a range of 5 minutes to 60 minutes, or 8 minutes to 45 minutes, or 12 minutes to 30 minutes, or can be about 15 minutes. In some embodiments, the predetermined frequency for running the vehicle or connecting the charger can be every day, every other day, every week, or the like.

In various embodiments the rewards matrix criteria 1200 can include event types pertinent to compliance with intoxication testing protocols. Such event types can include events that incentivize the user avoid substance use according to the desired parameters of the intoxication monitor system. In some embodiments, the user's action meets the reward matrix criteria if the user's action includes providing a sample each time an intoxication test is required. For instance, the user's action meets the reward matrix criteria if the user provides a sample each time the intoxication monitoring system prompts for a daily test.

In some embodiments, the user's action meets the reward matrix criteria if the user's action includes providing a sample each time an intoxication test is required for a predetermined duration. For instance, the user's action meets the reward matrix criteria if the user provides a sample each time the intoxication monitoring system prompts for a daily test over the span of a week, a month, a year, or the like.

In some embodiments, the user's action meets the reward matrix criteria if the user's action includes providing passing samples to intoxication tests. In some embodiments, a passing sample is a sample that has an intoxication level of zero (e.g., having a BAC of 0.000). Alternatively, a passing sample can be a sample that has an intoxication level that is below a threshold value (e.g., below the legal intoxication limit for driving).

In some embodiments, the user's action meets the reward matrix criteria if the user's action includes providing only passing samples for a predetermined duration. For instance, the user's action meets the reward matrix criteria if the user provides only passing samples for a daily test over the span of a week, a month, a year, or the like.

In some embodiments, the user's action meets the reward matrix criteria if the user's action includes providing less than a threshold amount of failing samples for a predetermined duration. For instance, the user's action meets the reward matrix criteria if the user provides less than a threshold amount of failing samples over the span of a week, a month, a year, or the like. The threshold amount of failing samples can be set by the intoxication mooting system. In some embodiments, the threshold amount of failing samples can be less than five, four, three, two, or one failing samples withing the predetermined time period.

In various embodiments the rewards matrix criteria 1200 can include event types pertinent to compliance with calibration. Such event types can include events that incentivize the user to calibrate the intoxication monitoring device 106 at an appropriate frequency. Calibration refers to the process of comparing and adjusting the device readings against a standard to maintain its accuracy over time. Intoxication monitoring devices could be subject to drift or variation in their sensory components due to environmental factors, sensor aging, or other operational conditions, which can impact reading precision. Accordingly, calibration of the intoxication monitoring device increases its reliability and accuracy. Moreover, the calibration procedure can be incorporated into the device maintenance logs and tracked as part of a user's compliance protocols. In various embodiments, rewards or penalties may be associated with the timely calibration of devices. Specifically, systems may be programmed to notify users of upcoming calibration deadlines and to document completion of the calibration within designated timeframes, as a means to encourage proactive maintenance behaviors.

In some embodiments, the user's action meets the reward matrix criteria if the user's action includes calibrating the intoxication monitoring device 106 within a specified time period. The specified time period can be set by the intoxication monitoring system 100. In some embodiments, the user's action meets the reward matrix criteria if they calibrate the intoxication monitoring device 106 within a time period of less than or equal to 30 days, 25 days, 20 days, 15 days, 10 days, 5 days, or 1 day, or an amount falling within a range between any of the foregoing.

In various embodiments the rewards matrix criteria 1200 can include event types pertinent to correctly using the intoxication monitoring system. In some embodiments, the user's action meets the reward matrix criteria if the user's action includes demonstrating proficiency in the intoxication monitoring device's required breath pattern.

In various embodiments, the intoxication monitoring system can incorporate a training module to assist the user in learning how to provide an appropriate breath sample to the intoxication monitoring device 106. In some embodiments the training module is designed to present the user with a series of progressively challenging instructional sessions that simulate the operational requirements of the intoxication monitoring device. During these sessions, the user is instructed to breathe into the intoxication monitoring device 106 and receives real-time feedback on their performance through visual and/or audible prompts displayed on the mobile device interface. Such prompts may include graphical representations such as a floating ball or bar indicators, visually contrasting the user's breath pattern against the device's ideal threshold. Utilizing sensor technologies embedded within the mobile device, such as microphones and accelerometers, the system is able to collect data regarding the breath sample provided by the user. This data can then be analyzed and compared to the standard device specifications to ascertain the degree of conformity with the required breath pattern.

In various embodiments, the intoxication monitoring system is configured to incentivize the user to engage with the training module. In some embodiments, the user's action meets the reward matrix criteria if the user's action includes engaging with the training module a predetermined number of times. In some embodiments, the user's action meets the reward matrix criteria if the user's action includes completing the training module. In various embodiments, the user can complete the training module by demonstrating consistent proficiency in providing a breath sample to the intoxication monitoring device 106.

In various embodiments the rewards matrix criteria 1200 can include event types pertinent to engaging with the system. For instance, the rewards matrix criteria 1200 can incentivize the user to engage with a mobile application associated with the intoxication monitoring system. In some embodiments, the user's action meets the reward matrix criteria if the user's action includes engaging with the mobile application for a predetermined number of consecutive days. In some embodiments, the user's action meets the reward matrix criteria if the user's action includes demonstrating continuous use of the user interface 316.

In various embodiments, the rewards matrix criteria 1200 can incentivize the user to continue using the intoxication monitoring system after they are legally required to do so. In some embodiments, the user's action meets the reward matrix criteria if the user's action includes utilizing the intoxication monitoring device beyond any legal requirements to do so.

In various embodiments, the rewards matrix criteria 1200 can incentivize the user to self-report information to the intoxication monitoring system. Such information can include information regarding the user's substance use, information regarding user's experience with the intoxication monitoring system, or the like. In some embodiments, the user's action meets the reward matrix criteria if the user's action includes using the user interface 316 self-reporting information.

In various embodiments the rewards matrix criteria 1200 can include event types pertinent retest protocols. In various embodiments, the intoxication monitoring system is configured to initiate a retesting protocol upon certain triggering events, such as an initial test result indicating intoxication, if the user fails to complete a test within a designated timeframe, or if a predetermined amount of time has elapsed since the user's last test. In some embodiments, the user's action meets the reward matrix criteria if the user's action includes successfully completing a predetermined number of retests.

In some scenarios, a user may inadvertently miss a required retest when they fail to check the intoxication monitoring system before turning off their vehicle. In various embodiments, the rewards matrix criteria 1200 can incentivize the user to check for any outstanding retests before exiting the vehicle. In some embodiments, the user's action meets the reward matrix criteria if the user's action includes checking the intoxication monitoring device 106 prior to exiting a vehicle 104 to determine if a retest is required.

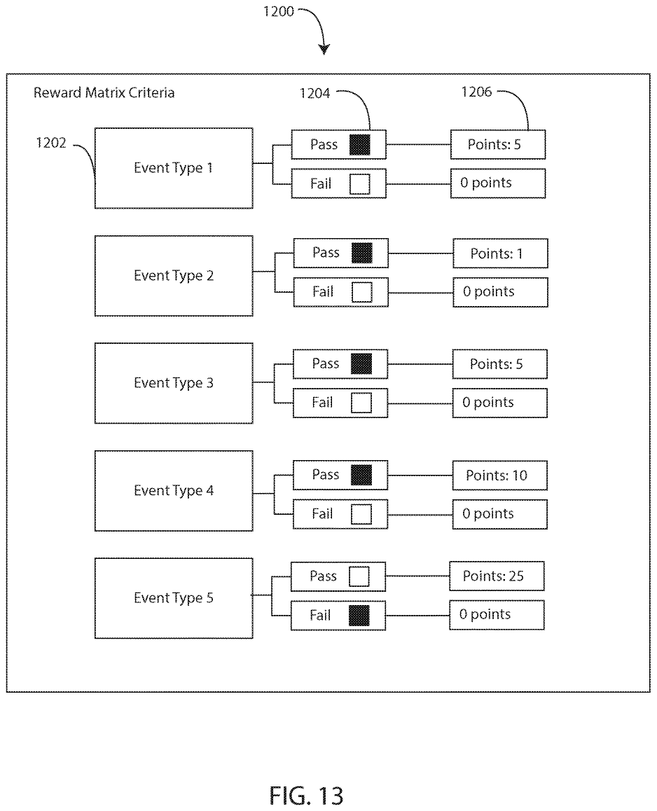

Referring now to FIG. 13, a schematic view of reward matrix criteria is shown in accordance with various embodiments herein. In the example of FIG. 13, a plurality of user actions have been applied to the rewards matrix criteria 1200. As described above, the user actions can be a plurality of log events from a device logs. In various embodiments, the intoxication monitoring system 100 is configured to compare elements of the reward matrix criteria with the user's actions to determine if the elements of the reward matrix criteria have been fulfilled.

In the example of FIG. 13, the user's actions have fulfilled the rewards criteria for event type 1. Accordingly, the user receives a “Pass” and is rewarded five points. Moreover, the user's actions have fulfilled the rewards criteria for event type 2. Accordingly, the user receives a “Pass” and is rewarded one point. Moreover, the user's actions have fulfilled the rewards criteria for event type 3. Accordingly, the user receives a “Pass” and is rewarded five points. Moreover, the user's actions have fulfilled the rewards criteria for event type 4. Accordingly, the user receives a “Pass” and is rewarded ten points. Moreover, the user's actions have not fulfilled the rewards criteria for event type 5. Accordingly, the user receives a “Fail” and is rewarded zero points.

Based on the user's actions relative to the reward matrix criteria, the intoxication monitoring system can change a point total associated with the user's account each time the user's action meets the reward matrix criteria. In some embodiments, changing a point total associated with the user's account includes increasing the user's point total by one or more points. In the example of FIG. 13, after analyzing event types 1-5, the system changes the point total associated with the user's account by increasing the user's point total by twenty-one points.

In various embodiments, the intoxication monitoring system is configured to alert the user to changes to their accumulated point total. In the example of FIG. 13, the user interface 316 can alert the user that their accumulated point total has increased by 15 points.

In various embodiments, the intoxication monitoring system is configured to inform the user on ways to increase their point total. For instance, the user interface 316 can provide a notification to the user if the user's action does not meet the reward matrix criteria. In some embodiments, the notification can include a recommendation to correct the user's action. In the example of FIG. 13, the user failed to meet the rewards criteria for event type 5 and the user interface 316 can include a recommendation on how to successfully meet the rewards criteria for event type 5.

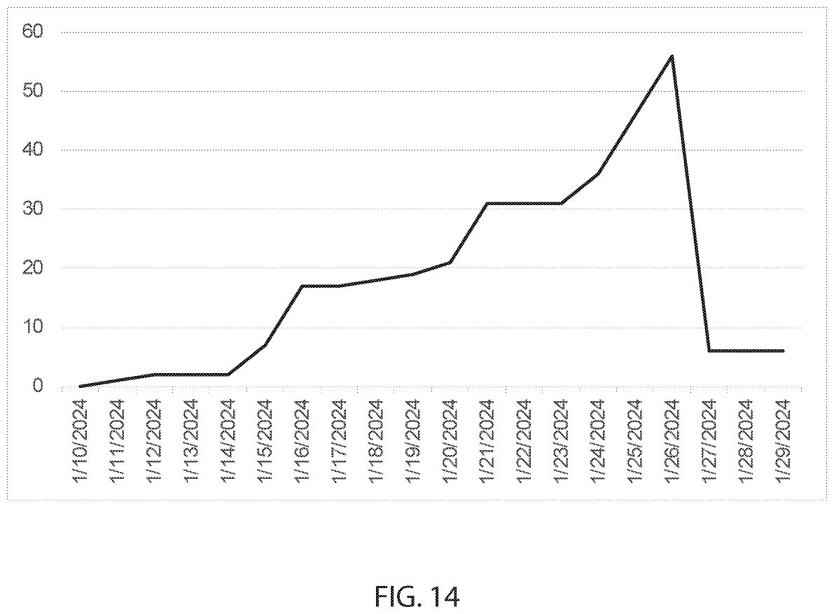

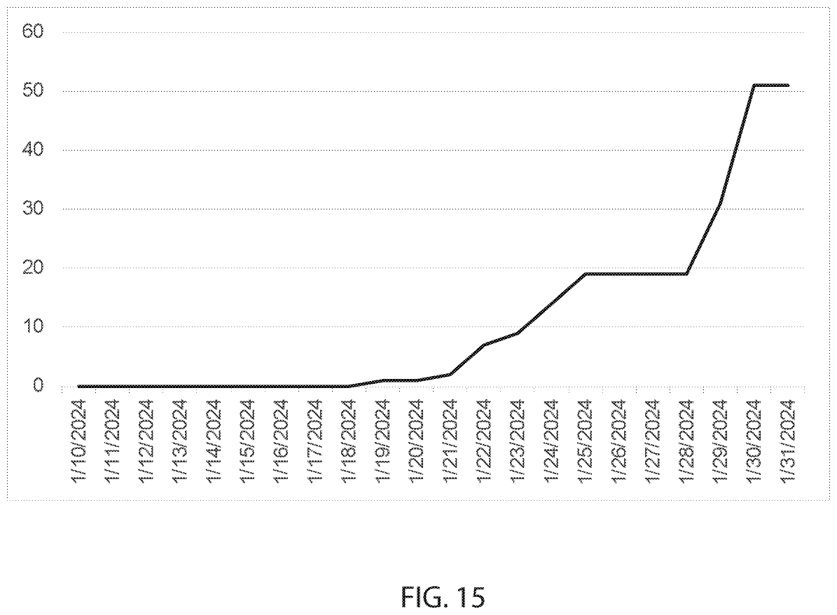

### Points and Redemption (FIGS. 14-15)

In various embodiments, the intoxication monitoring system 100 leverages a point-based system designed to encourage and reward users for consistent compliance and active engagement with the system. Upon successful completion of specific tasks or achievements, such as passing sobriety tests, maintaining a vehicle within prescribed parameters, or adhering to other predefined criteria, users receive points added to their respective accounts which directly influence their overall standing. In various embodiments a user can review their progress with a user interface 316, such as on a mobile device 112, where users can monitor their cumulative points and receive periodic updates regarding their progress.

In various embodiments, the user can redeem the points that their account has accumulated. The intoxication monitoring system can be configured to prompt the user when a redemption opportunity is available. For instance, as depicted by FIG. 4, the user interface 316 can display one or more redemption notifications 434 selectable by the user when the point total surpasses a redemption threshold.

In various embodiments, when a user redeems their points for one or more rewards, their accumulated points will decrease by the number of points spent. The system allows the point total associated with the user's account to dynamically decrease during certain interactions, particularly in instances where the user elects to redeem points for specific rewards. A visual representation of the current point total, indicative of the user's status level, is continuously available via the user interface to ensure real-time tracking.

In various embodiments, the user interface 316 can be further configured to present various reward options that are available for redemption. Redemption options can encompass an array of services and benefits. In some embodiments, the rewards can enable the user to save on the costs associated with maintaining the intoxication monitoring system. Such rewards can include complimentary roadside assistance, insurance coverage for the intoxication monitoring device, free or discounted device upgrades, bill credits free or discounted device servicing, free installation of an ignition interlock system, a free month or other time period of intoxication monitoring service, a free month or other time period of ignition interlock service, or the like. Rewards can take any suitable form such as gift cards, accessories for the intoxication monitoring system, and items such as hats, mugs, sunglasses, or the like.

In various embodiments, redemption options can further encompass an array of in-app benefits. In some embodiments, the rewards can enable users to unlock any of new features of the app, new customizations of the app (e.g., background colors), new avatars, or the like. In some embodiments, the rewards can enable the user to use the app more efficiently. For instance, the user can redeem rewards to remove ads from the app, skip over previously required training sessions, or the like.

In various embodiments, the intoxication monitoring system can have a plurality of status levels, and the user can use their accumulated points to increase their status level with the intoxication monitoring system. In various embodiments, the user's total points can be indicative of a user's status level. For instance, a user with more points will have a higher status level than a user with less points.

In various embodiments, once a user obtains a sufficient number of points, their status level will increase to the next highest level. In various embodiments, the user interface 316 can display one or more notifications indicating an increase in a status level of the user once enough points are received.

In various embodiments, the redemption notification is indicative of the user's status level. As the user's status level increases, they can enjoy more privileges and/or benefits. In some embodiments, higher status levels can unlock rewards that are not available to users at lower status levels. In some embodiments, when the user reaches a higher status level, they can receive rewards without redeeming any points such as free device insurance, or the like. In some embodiments, when the user reaches a higher status level, they can gain privileges with the intoxication monitoring system. For instance, upon reaching a higher status level, the intoxication monitoring system may require less frequent intoxication tests.

In various embodiments, the user interface 316 can additionally display the points accumulated by other users. For instance, the user interface 316 can anonymously display the points accumulated by other users in the same geographic area as the user. Such a configuration can allow the user to see how well they are performing relative to their peers. The system can allow multiple users to compete and distribute additional rewards to higher performing users.

Referring now to FIG. 14, a graph depicting a user's accumulated points over time is shown in accordance with various embodiments herein. FIG. 14 depicts a visual representation of a user's point total over time. Such a visual representation can be generated on the user interface 316 to enable the user to track their points and status level over time.

The example of FIG. 14 spans a time frame from Jan. 10, 2024, to Jan. 29, 2024. On Jan. 10, 2024, the user first opens their account with the intoxication monitoring system. Upon first opening the account, the user has not accumulated any points. Accordingly, the graph shows the user having zero points on Jan. 10, 2024. As the user interacts with the intoxication monitoring system over the coming days, they accumulate points by meeting the rewards matrix criteria.

On Jan. 26, 2024, the user has 55 points. The user is presented with one or more redemption notifications on the user interface. The user selects a reward costing 50 points. The intoxication monitoring system then receives and processes the user's redemption selection, and the points are withdrawn from the user's account. This can be seen by the user's accumulated points dropping from 55 to 5 points after redeeming their reward on Jan. 26, 2024.

Referring now to FIG. 15, a graph depicting a user's accumulated points over time is shown in accordance with various embodiments herein. FIG. 15 depicts a visual representation of a suer's pont total over time. Such a visual representation can be generated on the user interface 316 to enable the user to track their points and status level over time.

The example of FIG. 15 spans a time frame from Jan. 10, 2024, to Jan. 29, 2024. On Jan. 10, 2024, the user first opens their account intoxication monitoring system. Upon first opening the account, the user has not accumulated any points. Accordingly, the graph shows the user having zero points on Jan. 10, 2024.

As the user interacts with the intoxication monitoring system over the coming days, they accumulate points by meeting the rewards matrix criteria. In the example of FIG. 15, the user begins earning rewards points on Jan. 18, 2024. The user continues to accumulate points until Jan. 31, 2024. Unlike the example of FIG. 14, the user does not redeem any points, so the accumulated points associated with the user's account continues to increase.

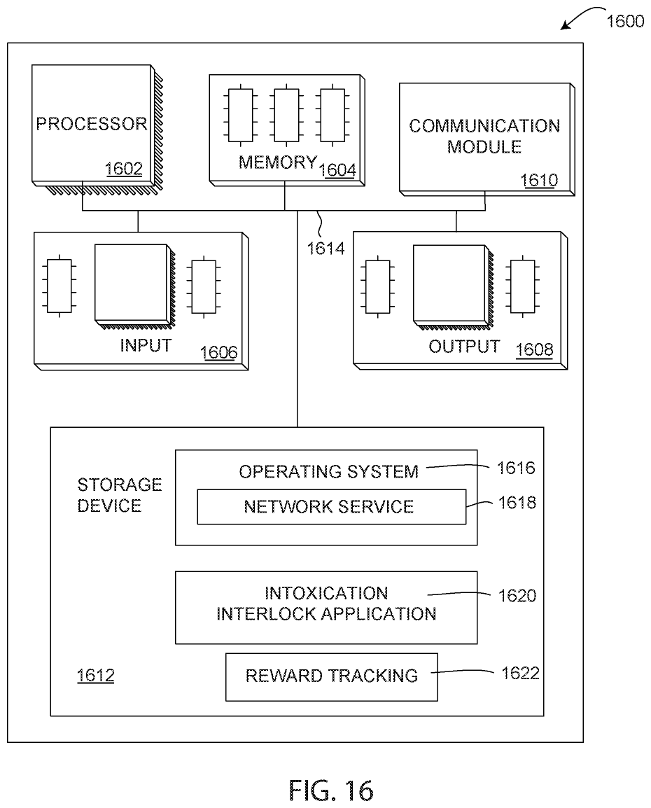

### Computer System (FIG. 16)

Referring now to FIG. 16, a computer system is shown in accordance with various examples herein. FIG. 16 illustrates one example of a computing device 1600, but it should be understood that additional computing devices 1600 may be used in other embodiments, such as discussed in regard to FIG. 5. Although computing device 1600 is shown as a standalone computing device, computing device 1600 may be any component or system that includes one or more processors or another suitable computing environment for executing software instructions in other examples and need not include all of the elements shown here. A portion of a subject testing device, such as a control system, a wireless relay system, and an intoxication monitoring device as described herein are examples of components that can be implemented using computing devices such as computing device 1600. In other embodiments, the mobile device 112 and/or the server 114 can include a computer system 1600.

As shown in the embodiment of FIG. 16, computing device 1600 includes one or more processors 1602, memory 1604, one or more input devices 1606, one or more output devices 1608, one or more communication modules 1610, and one or more storage devices 1612. Computing device 1600, in one example, includes an operating system 1616 executable by computing device 1600. The operating system includes various examples services such as a network service 1618. One or more applications, such as an intoxication interlock application 1620, are also stored on storage device 1612 and are executable by computing device 1600.

Each of components 1602, 1604, 1606, 1608, 1610, and 1612 may be interconnected (physically, communicatively, and/or operatively) for inter-component communications, such as via one or more communications channels 1614. In some examples, communication channels 1614 include a system bus, network connection, inter-processor communication network, or any other channel for communicating data. Applications such as intoxication interlock application 1620 and operating system 1616 may also communicate information with one another as well as with other components in computing device 1600.

Processors 1602, in one example, are configured to implement functionality and/or process instructions for execution within computing device 1600. For example, processors 1602 may be capable of processing instructions stored in storage device 1612 or memory 1604. Examples of processors 1602 include any one or more of a microprocessor, a controller, a digital signal processor (DSP), an application specific integrated circuit (ASIC), a field-programmable gate array (FPGA), or similar discrete or integrated logic circuitry.

One or more storage devices 1612 may be configured to store information within computing device 1600 during operation. Storage device 1612, in some examples, is known as a computer-readable storage medium. In some examples, storage device 1612 comprises temporary memory, meaning that a primary purpose of storage device 1612 is not long-term storage. Storage device 1612 in some embodiments includes a volatile memory, meaning that storage device 1612 does not maintain stored contents when computing device 1600 is turned off. In other embodiments, data is loaded from storage device 1612 into memory 1604 during operation. Examples of volatile memories include random access memories (RAM), dynamic random-access memories (DRAM), static random-access memories (SRAM), and other forms of volatile memories known in the art. In some examples, storage device 1612 is used to store program instructions for execution by processors 1602. Storage device 1612 and memory 1604, in various embodiments, are used by software or applications running on computing device 1600 such as intoxication interlock application 1620 and/or a reward tracking application 1622 to temporarily store information during program execution.

Storage device 1612, in some examples, includes one or more computer-readable storage media that may be configured to store larger amounts of information than volatile memory. Storage device 1612 may further be configured for long-term storage of information. In some examples, storage devices 1612 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

Computing device 1600, in some examples, also includes one or more communication modules 1610. Computing device 1600 in one example uses communication module 1610 to communicate with external devices via one or more networks, such as one or more wireless networks. Communication module 1610 may be a network interface card, such as an Ethernet card, an optical transceiver, a radio frequency transceiver, or any other type of device that can send and/or receive information. Other examples of such network interfaces include Bluetooth, 3G, 4G, 5G, Wi-Fi radios, and Near-Field Communications (NFC), and Universal Serial Bus (USB). In some examples, computing device 1600 uses communication module 1610 to wirelessly communicate with an external device such as via public network such as the Internet.

Computing device 1600 also includes in one example one or more input devices 1606. Input device 1606, in some examples, is configured to receive input from a user through tactile, audio, or video input. Examples of input device 1606 include a touchscreen display, a mouse, a keyboard, a voice responsive system, video camera, microphone, or any other type of device for detecting input from a user. Sensors, such as temperature sensors or vibration sensors, can also be included in the device 1600 or communicate with the device 1600 to provide information to a processor. For example, a vibration sensor or temperature sensor can be configured to provide information about a vehicle engine, such as whether or not it is running. A wireless transceiver can act as an input device 1606 when it is configured to provide feedback confirming that instructions are received or actions are executed.

One or more output devices 1608 may also be included in computing device 1600. Output device 1608, in some examples, is configured to provide output to a user using tactile, audio, or video stimuli. Output device 1608, in one example, includes a display, a sound card, a video graphics adaptor card, or any other type of device for converting a signal into an appropriate form understandable to humans or machines. Additional examples of output device 1608 include a speaker, a light-emitting diode (LED) display, a liquid crystal display (LCD), or any other type of device that can generate output to a user.

Computing device 1600 may include operating system 1616. Operating system 1616, in some examples, controls the operation of components of computing device 1600, and provides an interface from various applications such intoxication interlock application 1620 to components of computing device 1600. For example, operating system 1616, in one example, facilitates the communication of various applications such as intoxication interlock application 1620 with processors 1602, communication unit 1610, storage device 1612, input device 1606, and output device 1608. Applications such as intoxication interlock application 1620 may include program instructions and/or data that are executable by computing device 1600. As one example, intoxication interlock application 1620 may include instructions that cause computing device 1600 to perform one or more of the operations and actions described in the examples presented herein.

Although specific embodiments have been illustrated and described herein, any arrangement that achieve the same purpose, structure, or function may be substituted for the specific embodiments shown. This application is intended to cover any adaptations or variations of the example embodiments of the invention described herein. These and other embodiments are within the scope of the following claims and their equivalents.

### Intoxication Monitoring Device Options

Throughout the application, an intoxication monitoring device has been described. In some embodiments, the intoxication monitoring device can be a part of a vehicle immobilization system. In some embodiments, the intoxication monitoring device can be a part of a monitoring system, such as a portable monitoring system or an at-home monitoring system. In various embodiments, the intoxication monitoring device can be configured to receive a biological sample from a user. In some embodiments, the biological sample can include a breath sample, a blood sample, a saliva sample, a sweat sample, or a urine sample.

The intoxication monitoring device has sometimes been described as an alcohol breath detector which a user blows into, causing the breath sample to enter a fuel cell detector element. In alternative methods and systems, different intoxication monitoring devices and detection elements can be used.

For example, instead of or in addition to a fuel cell detection element, the system can include one or more of a complementary metal oxide semiconductor (CMOS) sensor, a metal oxide semiconductor (MOS) sensor, a semiconductor sensor, and an infrared (IR) sensor.

In some examples, detection elements can be used that provide a continuous signal. Detection elements for alcohol that can provide a continuous signal are a complementary metal oxide semiconductor (CMOS) sensor, a metal oxide semiconductor (MOS) sensor, a semiconductor sensor, and an infrared (IR) sensor. These detection elements and detection elements that provide a continuous signal do not need to wait for a predetermined time period after receiving a gas sample to provide a gas alcohol content reading that is considered accurate. In contrast, fuel cell detection elements operate on a fixed volume gas sample when measuring alcohol content.

In other examples, instead of testing a user's breath for alcohol, the intoxication monitoring device can test the user's breath for cannabis (i.e. tetrahydrocannabinol), opioids, or other intoxicants. In other examples, instead of testing the user's breath for an intoxicant, the intoxication monitoring device can be a transdermal device that is in contact with the user's skin and detects a level of alcohol, cannabis, opioids, or other intoxicants in a user. Transdermal sensors can be part of a wrist-worn device, an ankle-worn device, a device attached to another part of the user's body, or a device embedded in a steering wheel or another part of the vehicle. If a transdermal unit is used as the intoxication monitoring device, then instead of prompting the user to provide a breath sample, the system can ask the user to bring the intoxication monitoring device into contact with the user's skin. If the intoxication monitoring device is already in contact with the user's skin, then the system can simply take the measurement and determine if there is an intoxicant present above a threshold.

These alternatives for an intoxication monitoring device and a detection element can be used with each of the systems described herein instead of the breath detection element that is otherwise described. The systems and methods presented here may be implemented in part using a computerized device, such as a smartphone, handheld, or other computerized device.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An intoxication monitoring system comprising:
an intoxication monitoring device configured to receive and transmit data regarding a user's actions;
non-transitory computer-readable instructions executable by a processor, wherein execution of the instructions performs a method, the method comprising:
receiving the data regarding the user's actions, wherein the data identifies a user account associated with the intoxication monitoring device;
analyzing the user action by applying a reward matrix criteria;
changing a point total associated with the user's account when the user's action meets the reward matrix criteria;
displaying, on a user interface, one or more redemption notifications selectable by the user when the point total surpasses a redemption threshold;
receiving the user's redemption selection; and
processing the redemption selection.

2. The intoxication monitoring system of claim 1, wherein the intoxication monitoring device is an interlock device.

3. The intoxication monitoring system of claim 1, wherein the user interface is the user interface of a mobile device.

4. The intoxication monitoring system of claim 1, wherein applying the reward matrix criteria comprises comparing elements of the reward matrix criteria with the user's actions to determine if the elements of the reward matrix criteria have been fulfilled.

5. The intoxication monitoring system of claim 2, wherein the user's action meets the reward matrix criteria if the user's action comprises maintaining a suitable vehicle battery level for a specific amount of time or checking the intoxication monitoring device prior to exiting a vehicle to determine if a retest is required.

6. The intoxication monitoring system of claim 2, wherein the user's action meets the reward matrix criteria if the user's action comprises maintaining a suitable vehicle battery level for a specified time period.

7. The intoxication monitoring system of claim 6, wherein the vehicle battery level is considered suitable if the vehicle runs for at least 15 minutes every other day or if the vehicle battery is attached to a charger.

8. The intoxication monitoring system of claim 2, wherein the data transmitted from the intoxication monitoring device comprises the user's vehicular speed, location, vehicle battery voltage, and test results.

9. The intoxication monitoring system of claim 1, wherein the user's action meets the reward matrix criteria, if the user's action comprises demonstrating proficiency in the intoxication monitoring device's required breath pattern, calibrating of the intoxication monitoring device within a specified time period, providing a passing sample to the intoxication monitoring device, utilizing the intoxication monitoring device beyond any legal requirements to do so, using the user interface self-reporting information, or demonstrating continuous use of the user interface.

10. The intoxication monitoring system of claim 1, wherein the intoxication monitoring device is a handheld monitoring device.

11. The intoxication monitoring system of claim 1, wherein the user interface provides a notification to the user if the user's action does not meet the reward matrix criteria, wherein the notification comprises a recommendation to correct the user's action.

12. The intoxication monitoring system of claim 1, wherein the data transmitted from the intoxication monitoring device comprises the user's test results and location.

13. The intoxication monitoring system of claim 1, wherein changing a point total associated with the user's account comprises increasing the user's point total by one or more points.

14. The intoxication monitoring system of claim 1, wherein the method further comprises changing the point total associated with the user's account by decreasing the user's point total by one or more points in response to a redemption selection.

15. The intoxication monitoring system of claim 1, wherein the user interface is configured to display the user's total points.

16. The intoxication monitoring system of claim 1, wherein the user's total points are indicative of a user's status level.

17. The intoxication monitoring system of claim 1, wherein the redemption notification is indicative of the user's status level.

18. The intoxication monitoring system of claim 1, wherein the redemption notification comprises at least one of free roadside service, free intoxication monitoring device insurance, free accessories for the intoxication monitoring device, a bill credit, a gift card, free uninstallation of the intoxication monitoring device, or upgrades to the user interface.

19. A method of interacting with a user of an intoxication system to support reliable function, the method comprising:
receiving, from an intoxication monitoring device, a data regarding the user's action, wherein the data identifies a user account associated with the intoxication monitoring device;
analyzing, using a mobile device, the user's action by applying a reward matrix criteria;
changing a point total associated with the user's account if the user's actions meet the reward matrix criteria;
displaying, on the user interface of the mobile device, one or more redemption notifications selectable by the user once the point total is above a points threshold;
receiving the user's redemption selection; and
processing the redemption selection.

20. An intoxication monitoring system comprising:
an intoxication monitoring device configured to receive and transmit data regarding a user's action;
a mobile device in electronic communication with the intoxication monitoring device, the mobile device comprising:
a user interface;
a processor;
a memory coupled to the processor; and
non-transitory computer-readable instructions executable by the processor, wherein execution of the instructions causes the mobile device to perform a method, the method comprising:
receiving the data regarding the user's action, wherein the data identifies a
user account associated with the intoxication monitoring device;
analyzing the user's action by applying a reward matrix criteria;
changing a point total associated with the user's account if the user's action meet the reward matrix criteria;
displaying, on the user interface, one or more notifications indicating an increase in a status level of the user once enough points are received.
